SEQUENCE LISTING (24 sequences)

SEQ ID NO: 1 - DNA, 20 bp, Artificial Sequence
  HMGA2 forward primer
aagccactggagaaaaacgg

SEQ ID NO: 2 - DNA, 21 bp, Artificial Sequence
  HMGA2 reverse primer
ctcttcggcagactcttgtga

SEQ ID NO: 3 - DNA, 24 bp, Artificial Sequence
  PLAG1 forward primer
tgcttcattctgtgacggtctatt

SEQ ID NO: 4 - DNA, 24 bp, Artificial Sequence
  PLAG1 reverse primer
actttgatcttagccagtcccatt

SEQ ID NO: 5 - DNA, 19 bp, Artificial Sequence
  hKLK7 forward primer
ccctgctcagtggcaatca

SEQ ID NO: 6 - DNA, 18 bp, Artificial Sequence
  hKLK7 reverse primer
ctgtcgcccagcgtatca

SEQ ID NO: 7 - DNA, 20 bp, Artificial Sequence
  hFNDC4 forward primer
tcactcacctcagagccaac

SEQ ID NO: 8 - DNA, 19 bp, Artificial Sequence
  hFNDC4 reverse primer
ttcacctcccgaatcacac

SEQ ID NO: 9 - DNA, 20 bp, Artificial Sequence
  CDH3 forward primer
catcagcgtcatctccagtg

SEQ ID NO: 10 - DNA, 20 bp, Artificial Sequence
  CDH3 reverse primer
atcagtgaccgtcagcctct

SEQ ID NO: 11 - DNA, 22 bp, Artificial Sequence
  TPO1 forward primer
acaacagagaccaccccagatg

SEQ ID NO: 12 - DNA, 22 bp, Artificial Sequence
  TPO1 reverse primer
gccatcaggaggtcagaatagc

SEQ ID NO: 13 - DNA, 4150 bp, Homo sapiens
cttgaatcttggggcaggaactcagaaaacttccagcccgggcagcgcgcgcttggtgcaagactcaggagctagcagcccgtccccctccgactctccggtgccgccgctgcctgctcccgccaccctaggaggcgcggtgccacccactactctgtcctctgcctgtgctccgtgcccgaccctatcccggcggagtctccccatcctcctttgctttccgactgcccaaggcactttcaatctcaatctcttctctctctctctctctctctctctctctctctctctctctctctctctctctctcgcagggtggggggaagaggaggaggaattctttccccgcctaacatttcaagggacacaattcactccaagtctcttccctttccaagccgcttccgaagtgctcccggtgcccgcaactcctgatcccaacccgcgagaggagcctctgcgacctcaaagcctctcttccttctccctcgcttccctcctcctcttgctacctccacctccaccgccacctccacctccggcacccacccaccgccgccgccgccaccggcagcgcctcctcctctcctcctcctcctcccctcttctctttttggcagccgctggacgtccggtgttgatggtggcagcggcggcagcctaagcaacagcagccctcgcagcccgccagctcgcgctcgccccgccggcgtccccagccctatcacctcatctcccgaaaggtgctgggcagctccggggcggtcgaggcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgccaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggactatattaatcaccttctttgtaatcccttcacagtcccaggtttagtgaaaaactgctgtaaacacaggggacacagcttaacaatgcaacttttaattactgttttcttttttcttaacctactaatagtttgttgatctgataagcaagagtgggcgggtgagaaaaaccgaattgggtttagtcaatcactgcactgcatgcaaacaagaaacgtgtcacacttgtgacgtcgggcattcatataggaagaacgcggtgtgtaacactgtgtacacctcaaataccaccccaacccactccctgtagtgaatcctctgtttagaacaccaaagataaggactagatactactttctctttttcgtataatcttgtagacacttacttgatgatttttaactttttatttctaaatgagacgaaatgctgatgtatcctttcattcagctaacaaactagaaaaggttatgttcatttttcaaaaagggaagtaagcaaacaaatattgccaactcttctatttatggatatcacacatatcagcaggagtaataaatttactcacagcacttgttttcaggacaacacttcattttcaggaaatctacttcctacagagccaaaatgccatttagcaataaataacacttgtcagcctcagagcatttaaggaaactagacaagtaaaattatcctctttgtaatttaatgaaaaggtacaacagaataatgcatgatgaactcacctaattatgaggtgggaggagcgaaatctaaatttcttttgctatagttatacatcaatttaaaaagcaaaaaaaaaaaaggggggggcaatctctctctgtgtctttctctctctctcttcctctccctctctcttttcattgtgtatcagtttccatgaaagacctgaataccacttacctcaaattaagcatatgtgttacttcaagtaatacgttttgacataagatggttgaccaaggtgcttttcttcggcttgagttcaccatctcttcattcaaactgcacttttagccagagatgcaatatatccccactactcaatactacctctgaatgttacaacgaatttacagtctagtacttattacatgctgctatacacaagcaatgcaagaaaaaaacttactgggtaggtgattctaatcatctgcagttctttttgtacacttaattacagttaaagaagcaatctccttactgtgtttcagcatgactatgtatttttctatgtttttttaattaaaaatttttaaaatacttgtttcagcttctctgctagatttctacattaacttgaaaattttttaaccaagtcgctcctaggttcttaaggataattttcctcaatcacactacacatcacacaagatttgactgtaatatttaaatattaccctccaagtctgtacctcaaatgaattctttaaggagatggactaattgacttgcaaagacctacctccagacttcaaaaggaatgaacttgttacttgcagcattcatttgttttttcaatgtttgaaatagttcaaactgcagctaaccctagtcaaaactatttttgtaaaagacatttgatagaaaggaacacgtttttacatacttttgcaaaataagtaaataataaataaaataaaagccaaccttcaaagaaacttgaagctttgtaggtgagatgcaacaagccctgcttttgcataatgcaatcaaaaatatgtgtttttaagattagttgaatataagaaaatgcttgacaaatattttcatgtattttacacaaatgtgatttttgtaatatgtctcaaccagatttattttaaacgcttcttatgtagagtttttatgcctttctctcctagtgagtgtgctgactttttaacatggtattatcaactgggccaggaggtagtttctcatgacggcttttgtcagtatggcttttagtactgaagccaaatgaaactcaaaaccatctctcttccagctgcttcagggaggtagtttcaaaggccacatacctctctgagactggcagatcgctcactgttgtgaatcaccaaaggagctatggagagaattaaaactcaacattactgttaactgtgcgttaaataagcaaataaacagtggctcataaaaataaaagtcgcattccatatctttggatgggccttttagaaacctcattggccagctcataaaatggaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctacataagactggacatttaacttttctaccatttctgcaagttaggtatgtttgcaggagaaaagtatcaagacgtttaactgcagttgactttctccctgttcctttgagtgtcttctaactttattctttgttctttatgtagaattgctgtctatgattgtactttgaatcgcttgcttgttgaaaatatttctctagtgtattatcactgtctgttctgcacaataaacataacagcctctgtgatccccatgtgttttgattcctgctctttgttacagttccattaaatgagtaataaagtttggtcaaaacagaaaaaaaaaaa

SEQ ID NO: 14 - DNA, 360 bp, Homo sapiens
aagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgccaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggac

SEQ ID NO: 15 - DNA, 7311 bp, Homo sapiens
gcatacactacaatggctgctggaaagaggcgtaaggaaacaatttccaggcccgccgcgtccagcccgaaatatgagaaaaaaattattagaaattccgcgggcggtgtagaggcggcggacgggccggagggaggatgttaaagccccgcggttgcctcttggtgctgccttggccgtatttggcacccagaatgcttcattctgtgacggtctattaataaggttgccttgctagagtttggagcagggcctcagattggccaaaatgggaaggattggattccactctcttccacgaagagtcaatgggactggctaagatcaaagtctgaggctttttccatcagtaatcagtccctttttgctttcttttacgaccacatgaaacttgagaagccacctaaagctatatcatttagtggagttgggcagttcccaagtgtccaacaagaaggcctggtttaggctgcgatggccactgtcattcctggtgatttgtcagaagtaagagatacccagaaagtcccttcagggaaacgtaagcgtggtgaaaccaaaccaagaaaaaactttccttgccaactgtgtgacaaggcctttaacagtgttgagaaattaaaggttcactcctactctcacacaggagagaggccctacaagtgcatacaacaagactgcaccaaggcctttgtttctaagtacaaattacaaaggcacatggctactcattctcctgagaaaacccacaagtgtaattattgtgagaaaatgtttcaccggaaagatcatctgaagaatcacctccatacacacgaccctaacaaagagacgtttaagtgcgaagaatgtggcaagaactacaataccaagcttggatttaaacgtcacttggccttgcatgccgcaacaagtggtgacctcacctgtaaggtatgtttgcaaacttttgaaagcacgggagtgcttctggagcaccttaaatctcatgcaggcaagtcgtctggtggggttaaagaaaaaaagcaccagtgcgaacattgtgatcgccggttctacacccgaaaggatgtccggagacacatggtggtgcacactggaagaaaggacttcctctgtcagtattgtgcacagagatttgggcgaaaggatcacctgactcgacatatgaagaagagtcacaatcaagagcttctgaaggtcaaaacagaaccagtggatttccttgacccatttacctgcaatgtgtctgtgcctataaaagacgagctccttccggtgatgtccttaccttccagtgaactgttatcaaagccattcacaaacactttgcagttaaacctctacaacactccatttcagtccatgcagagctcgggatctgcccaccaaatgatcacaactttacctttgggaatgacatgcccaatagatatggacactgttcatccctctcaccacctttctttcaaatatccgttcagttctacctcatatgcaatttctattcctgaaaaagaacagccattaaagggggaaattgagagttacctgatggagttacaaggtggcgtgccctcttcatcccaagattctcaagcatcgtcatcatctaagctagggttggatcctcagattgggtccctagatgatggtgcaggagacctctccctatccaaaagctctatctccatcagtgaccccctaaacacaccagcattggatttttctcagttgtttaatttcatacctttaaatggtcctccctataatcctctatcagtggggagccttggaatgagctattcccaggaagaagcacattcttctgtttcccagctccccccacaaacacaggatcttcaggatcctgcaaacactatagggcttgggtctctgcactcactgtcagcagctttcaccagcagtttaagcacaagtaccaccctcccacgtttccatcaagcttttcagtaggattctgggacatggattcattacagaaatgtatgtgtagctgtgccctagatgaccatttttattttagtgcctactttaaaacagtataaaaatttctgcttttgtataatacaaattttcattaagccagtataaaatagaaactagcttttaaactgagctttggaaccatttgtgttcagttaagtttacctgggtattttgtcctgattcactgccaattgtcacattttaagacttttttttttccatataggaaagccattattagtagtaaacttttacaaatcccattttcaaattacttttagatcttaaaattttcatttttgtctaataacagtggctctaccttttgacatctggctcattaaaaaatttagcaatagaatgtaaattgtataaaaagtttgtgaataactcaagggtttaaattttcttactagcttctaaatggattaataatcaagtgcttcaaatgaattaagagtccagtttcggaagataataaatgtttgttagatacaccataatttcagatcagtatattctgaagactccctgttgtctggctaaaatatttgccatctttattatgagcctttaaggaaaacaaaccctaaacacaaagcatcagtatttatagcaaaaagagactctgttaggtgacatggcatttcgtgtcacttaatagttggccctaaattagtacacaggatattttgtcgtgtttcatccttcttaacatgctatcttttcatttaataatagtaatagtgtatggcattggggtcttcagagtcgatatataggtagatctctttagtcttttccacctttcacatccaaggggtgggtcaagtgcagccagcaatttattttcattgttggcccacggttagtccataatctagagccattgtggaactgcagccatgaggtgtgtttatcccacagtggattgactcagcctctgtgggtgacagacttctaagcaggaagatagacgtgaagcacatggttacatttgggaacttgtgtagggatcatggcccctgtagccagggttaaaaactggactttttagaagtaaagtaaaagcatatcgcttatatcatttcttgctgaatttgatatgtttttctttcccttaagaatcaaaagcagaaaacaaaaacaacagtcctactccgatgttatctttctgattcaatgtgaatccatctttccttgcaatattttggatggagaatttgaagttaaatgcattagaaaactacctgatgaactaccacaaagttttaagtgactagaaatatatacagtaaaatcccactttcatgcatctctgggaaatgataggagtattgcaaataagttgagtttgtagagggtaacaaagtaaagtaaaacaaacctatcttggttaacatgaaaataacaattgagaatatattatattcactgaataattataggcttttcctcacattagacaaccaacataatcttcttaaaggtctaattaatatatttttctaagggtcagttgggacattaacctaagaaacatatctattaagcacttgttaacaccttattttaggaccctttccgttggggatgggggcaagggtgggaggtttttagaagagtatatatctctttaaaaaaaaacagaaagaaaaatatttctgagcactcattagccctatatggaaacttctttcctttttgtagggccagttatcactgcagattgcaatgtttaccaagaatttctaaaaatgagtgcagattactgaatataatacattatttaaaatatttgggagtagtataatttgtggagaaatgtaaattgtaataatgtaaatgggggcttcaatatatatatataatacacacacacacacacatgcacacataccgcacttcatagaatcaaagttgctctctgaaggagctttggctcctgatattttatcatgctcctatatttttttaatccttggagcagtagtttttatacttatgtatttaaattttattatgaaaaattacatttattaaaaaagtgtgttccaaaggcattaaaattatatatgttaataaggaagtacatttttaaatttttcaaactgctcctagcttttgattaggagaatattttttctgaaagtaggcttttcgctctgcttcattactgcttcctttagtttctatgaaacagattgcttacctaaatctttagttgaatgattagtgttcaatattgctttaatcaccatataaaaggaaaaaaattggtgacagagcacaaatagaaaacctatttttaaatagaaatcacaaatagcaagtgtggaagcactactttattctgtttaaaatgtacttaagaagtcatcaaattagtgaactgagacattggccttagtaggctgtattcactgctaatttaaaaaagggagtaccaggatttattaagtaaagcattttggaaatggggaatagcgccatatatgtatgtatgtgtatgtgtgtgtgtgtgtgtgtatatatacacacacacacacatacttaaatcttgccctgcatgaaattcaaatacatggaggcacatcttcagggcaccagtgttaaaattttggagtcttaattttcatgtgtacacctctttgcctgttcccacccccagacttgaaataacacttcagagtaagagggaattcagctaatttgtttttaaaattgactgtagtggtcactaaaccctttttgagagaatttctattaaagatgaggcagactcgcttatttgaattgcacaatgttctaacaaggatgtaacacagaattggcttttttttccctagaaaaagattgtttgtttctatgtcaactagatatgattaaaaataagtattgccaatgctgttttcattctctagtggccagaatcattatccttgaaatttctggtagtgccttagcttggttaaaaaaaaaaaaaaaaaaaaaaaaaagggattaacattaaataaaagtagtttagaatttgggcctcagacaagatattgaacctcattcagtttcacttccacatgtatgtacaagttaggtcaccaaacacggaagtgtgagtgtggaaggatcttggcactgtaagcaatgctatccattgatgtatacaagtacctttatagttatcgatcactgttaaaactttcattttaaaatcctattaccaagttcagttttttaaaacttcaattgtcctggctgattatgcatcactctgtgtgcaacttttttatttcatttagtgtttctttcaagctgtgtatttttgcctatttgttgcttgtgctttatttttcttagtcatttgtggaatatagtgatatattgtgttaatttggacagtagcggtttttaaaaaccatatactgactgaaacatgagccagagccgattgctttattaagctaataatgaatgttaaagagtacatattttcaggatcgttcatctagtgagcaatacacatattataggccaatatttttttaaaaaatagagcttggtcaacctctatactacacatattacaagatatagcactttcaaaatgaatctaaacctttacagaaactttcttataggttatgccttttattttaagacttattataattcaagtgccattagatgatatatatgtaggcctttgatatataatgctttgtgtacaaaaatggtagatggtattttaaacaggtacatttttacagtgttttcttatcaatttgctatattgcacagaatcagtgtgtgtcttttcataaggttttacaatggtttatttttttacaaggtttacgtgtctcaaagcacactgtcttcccagtacgtaagttaaaaaataccagttcacccaagttgcttctagcctactgagatccatgtgacattggaggagatcttttaaatgtttagtattcgtcattagcaatggctggctgttagttctggtaaatgtgtgcctaagttgaatttgtcttgtttttctcacactgtgtcagcagccatgtctacaacacagataagtctgttgtgatcacatagatctacataagttgtgcagttttgtgctaaaaacccatagggagctcctttgggatcatagaaaagaagatcatgcaaccagcattggtgaaggcacactcagattgcacttagggcctttctatgatgttgtcaaccctctgaggatggaaggcagtgtcttttgatgttatctagcctagaaatgacacagaactattgctaatgtataaaacacttcattatataagcttcagtggtacagatgaaccagaatgaatgtttatcttctcagaaacactccttcaatattatattggatcatgctgctaatgtaacttgggctacaactcttcatggtgctacaaacttctctgtctcattcagtcgtatttttttatccatagaaaaaggactacattaggtgtaaaagtgtacaatatatttttatactgtgacttaatttgtcattaacaaacttttacaccaccacaatgtattcatgtgcacttgcaaaaggagatctcggacatgcaaatgttaccagaacaaacccagcttttgtccacaaggtgactgtaactcagaatggaaagtgggctttataatagggtgtggagtgaagaacatgctgtatgttactaacagccctttgaatttaacaaaaactgggaatccattaggaaacggattgcatcatacctgaacataagctggactgctgaaattgtatttttagctaatgaaaaagtgtttggactagtactctaaaaatgttctaatgataaagttttgagtcaaaatagaaaagaaaaaaatctgcattccaggccgaattttgtatatttttattgcatttaaaattgctattctgtaatattgggaaatcaagtggcttatcatgtatatcgtgtacttaaaatgtattcacaaactactgttgtatttgtataaaatatagacaaagatcatattttttgtgtgtgtataagctctgtaaaatagcaatcacattatgaagctgcagtgatactacattttaaacattcacatccaaagaagcagactatttattgtccatataccagatttaaaatattaatttgctgctaattaaataatagtactgcagcttcttgtggcctacagtgttatgtttgctgtaagaataagatatgtgaattccacaaaatatatgaataaaattatagaatggcttta

SEQ ID NO: 16 - DNA, 360 bp, Homo sapiens
gttgcctcttggtgctgccttggccgtatttggcacccagaatgcttcattctgtgacggtctattaataaggttgccttgctagagtttggagcagggcctcagattggccaaaatgggaaggattggattccactctcttccacgaagagtcaatgggactggctaagatcaaagtctgaggctttttccatcagtaatcagtccctttttgctttcttttacgaccacatgaaacttgagaagccacctaaagctatatcatttagtggagttgggcagttcccaagtgtccaacaagaaggcctggtttaggctgcgatggccactgtcattcctggtgatttgtcagaagtaaga

SEQ ID NO: 17 - DNA, 1955 bp, Homo sapiens
gaagattttggagcccagctgtgtgccagcccaagtcggaacttggatcacatcagatcctctcgagctccagcaggagaggcccttcctcgcctggcagcccctgagcggctcagcagggcaccatggcaagatcccttctcctgcccctgcagatcttactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcatggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaagatctaa

SEQ ID NO: 18 - DNA, 360 bp, Homo sapiens
ggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtc

SEQ ID NO: 19 - DNA, 1612 bp, Homo sapiens
gcagaaggcggcggctggctctccgggacggtcacatcccgctgcaggggcgggcggaggccgccgcactgcctcccgcaccggggacccaggccagcgtccgggcaacgccccctgctcccggacagactccgtggcccgctcgagccctgggggctccgcagacccgcgcccgctccgcccgcagctcggccccgcgctgcccgcgtcgccgggcccgcgccgggatggggtaggggcagcgccaccgagtcgggcgatgggccgccctctgggcaccgagcagccccccgaggcctgaccaaccgcgaggaccggcggaggagccccgcctggatgtcaagcggatgccaagcggatgccacagttccccccccagcggactccgtggggacatggcttcgctggtgcccctttccccatatctaagccccacggtcctcctgctggtcagctgtgacctgggcttcgtgcgagcagaccggcctccctctcctgtgaatgtgacggtcactcacctcagagccaactcggccactgtgtcctgggacgtcccagaaggcaacatcgtcattggctactccatttcccagcaacggcagaatggccccgggcagcgtgtgattcgggaggtgaacaccaccacccgggcctgtgccctctggggcctggctgaagacagtgactacacagtgcaggtcaggagcatcggccttcggggagagagtcccccagggccccgggtgcacttccgaactctcaagggttctgaccggctaccttcaaacagttcaagcccaggtgacatcacagtggaaggtctggatggagagcggccactgcagactggggaagtggtcatcattgtggtggtgttgctcatgtgggctgctgtaattgggctgttctgccgtcagtatgacatcatcaaggacaatgactccaacaacaatcccaaggagaagggaaaggggccggaacagagtcctcagggaaggccagtggggacaagacagaaaaagtcaccatctatcaacaccatcgacgtttgagtgaagaaacacacccagaagagagatgcactaacaactggggatagggatggggtcagggggagcccaagatggtgatctgcccgagactcccagagggtaatgccactcccacaatctcaggcctggtacccatcctctttccactgtgagcagagccagaaggtaggtctgttcagagtctgtgcccctggacctggggagtggatatcagatgggatatctccttccattccccggtccaggggagagtcactagttgtaccctactccattaggtcccaaatgggggccccatttcacctgtatcaggactctgagcatccccagctgccccacatcttgcctctggccctcagagaggggtgtttctgtgggtactcctcttaccccagcaaataaaaggaattgtctgaccctagaggcagatgctgcactgcactactccaatgtcttccatggagcctcaggtgctccccctctcacctggcagccccttcagctgctagtgatatcacttgttggacatttttccaataaaggttcttggacaaactgga

SEQ ID NO: 20 - DNA, 320 bp, Homo sapiens
accggcctccctctcctgtgaatgtgacggtcactcacctcagagccaactcggccactgtgtcctgggacgtcccagaaggcaacatcgtcattggctactccatttcccagcaacggcagaatggccccgggcagcgtgtgattcgggaggtgaacaccaccacccgggcctgtgccctctggggcctggctgaagacagtgactacacagtgcaggtcaggagcatcggccttcggggagagagtcccccagggccccgggtgcacttccgaactctcaagggttctgaccggctaccttcaaacagttcaagccca

SEQ ID NO: 21 - DNA, 4452 bp, Homo sapiens
aaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctcggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggtggcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccagaggtgcctggggagtgaactgttttctaaatagaaggtttattggcatctaactcacataccataccattcacttgtttaacgtttacaattcaatggtttttagaattttcagagttctgcaaaaagagtgggctctgtttaccctgctggcttcacccaagcttcctctcaatggcaggggatactcagggtcagcttccatgcctaagtgggctcagggaggggagacttgccctcctttgcaaagtcagttgctccatgagagagaaccattaacctcttaccccaaggctgagcccctccacagccccagcaaggtctcttctggaacagcggctgccctccctgcaggacagtgtgtgacgacttcctcttctatgctggaggggtccctgttgaaaggcaggggttggggaacagccagctctgctacttgctagcacatttttttttttttttttgagacggagtctcgttctgtcgcccaggctggagtgcagtggtgtgatcttggctcactgcaagctctgcctcctgggttcacgccattctcctgcctcagcctcccgagtagctgggactacaggtgcctgccactatgcccagctaattttttttgagtgtgtgtgtttttttagtagagacggggtttcactgtgttagccaggatgtctcgatctcctgacctcgtgatccgcccacgtcggcctcccaaagtgctaggattacaggcttgagccaccgcgcccagcgctggcacatcatttaacctccagttgcctcagattttacatttacaaaatggggagtttttgtggagattaagtgaattaatatctggcacatggtccatgcaatctgttagttggtaacagctaccatttattcagtacttttaaaggccagacaggacttcgattatttcctctaaatcctcacaatcaccctctgaggggactttctcctttaaagaatggccacattgtatttgtttttttaaatgacatctggtcatcatcgaaatcaagcaaaacaaaattaagagaacctacccaagatgtcagtgaaattggaacattcctgacaataccagggcataaatgcaggaatcaggaataggcagcagtgatagaacaattctgtttgtgcccttgttaacgtgaagttcaaagtcatctttgcaattagccaaaagaatctgaagtgaagctgaggaaattgctgatgttgaaataaacatttccttccatga

SEQ ID NO: 22 - DNA, 480 bp, Homo sapiens
tctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaacc

SEQ ID NO: 23 - DNA, 3152 bp, Homo sapiens
ggaaggcaattaaggcgcccatttcagaagagttacagccgtgaaaattactcagcagtgcagttggctgagaagaggaaaaaaggtcagaatgagagcgctcgctgtgctgtctgtcacgctggttatggcctgcacagaagccttcttccccttcatctcgagagggaaagaactcctttggggaaagcctgaggagtctcgtgtctctagcgtcttggaggaaagcaagcgcctggtggacaccgccatgtacgccacgatgcagagaaacctcaagaaaagaggaatcctttctccagctcagcttctgtctttttccaaacttcctgagccaacaagcggagtgattgcccgagcagcagagataatggaaacatcaatacaagcgatgaaaagaaaagtcaacctgaaaactcaacaatcacagcatccaacggatgctttatcagaagatctgctgagcatcattgcaaacatgtctggatgtctcccttacatgctgcccccaaaatgcccaaacacttgcctggcgaacaaatacaggcccatcacaggagcttgcaacaacagagaccaccccagatggggcgcctccaacacggccctggcacgatggctccctccagtctatgaggacggcttcagtcagccccgaggctggaaccccggcttcttgtacaacgggttcccactgcccccggtccgggaggtgacaagacatgtcattcaagtttcaaatgaggttgtcacagatgatgaccgctattctgacctcctgatggcatggggacaatacatcgaccacgacatcgcgttcacaccacagagcaccagcaaagctgccttcgggggaggggctgactgccagatgacttgtgagaaccaaaacccatgttttcccatacaactcccggaggaggcccggccggccgcgggcaccgcctgtctgcccttctaccgctcttcggccgcctgcggcaccggggaccaaggcgcgctctttgggaacctgtccacggccaacccgcggcagcagatgaacgggttgacctcgttcctggacgcgtccaccgtgtatggcagctccccggccctagagaggcagctgcggaactggaccagtgccgaagggctgctccgcgtccacgcgcgcctccgggactccggccgcgcctacctgcccttcgtgccgccacgcgcgcctgcggcctgtgcgcccgagcccggcatccccggagagacccgcgggccctgcttcctggccggagacggccgcgccagcgaggtcccctccctgacggcactgcacacgctgtggctgcgcgagcacaaccgcctggccgcggcgctcaaggccctcaatgcgcactggagcgcggacgccgtgtaccaggaggcgcgcaaggtcgtgggcgctctgcaccagatcatcaccctgagggattacatccccaggatcctgggacccgaggccttccagcagtacgtgggtccctatgaaggctatgactccaccgccaaccccactgtgtccaacgtgttctccacagccgccttccgcttcggccatgccacgatccacccgctggtgaggaggctggacgccagcttccaggagcaccccgacctgcccgggctgtggctgcaccaggctttcttcagcccatggacattactccgtggaggtggtttggacccactaatacgaggccttcttgcaagaccagccaaactgcaggtgcaggatcagctgatgaacgaggagctgacggaaaggctctttgtgctgtccaattccagcaccttggatctggcgtccatcaacctgcagaggggccgggaccacgggctgccaggttacaatgagtggagggagttctgcggcctgcctcgcctggagacccccgctgacctgagcacagccatcgccagcaggagcgtggccgacaagatcctggacttgtacaagcatcctgacaacatcgatgtctggctgggaggcttagctgaaaacttcctccccagggctcggacagggcccctgtttgcctgtctcattgggaagcagatgaaggctctgcgggacggtgactggttttggtgggagaacagccacgtcttcacggatgcacagaggcgtgagctggagaagcactccctgtctcgggtcatctgtgacaacactggcctcaccagggtgcccatggatgccttccaagtcggcaaattccccgaagactttgagtcttgtgacagcatcactggcatgaacctggaggcctggagggaaacctttcctcaagacgacaagtgtggcttcccagagagcgtggagaatggggactttgtgcactgtgaggagtctgggaggcgcgtgctggtgtattcctgccggcacgggtatgagctccaaggccgggagcagctcacttgcacccaggaaggatgggatttccagcctcccctctgcaaagatgtgaacgagtgtgcagacggtgcccaccccccctgccacgcctctgcgaggtgcagaaacaccaaaggcggcttccagtgtctctgcgcggacccctacgagttaggagacgatgggagaacctgcgtagactccgggaggctccctcgggtgacttggatctccatgtcgctggctgctctgctgatcggaggcttcgcaggtctcacctcgacggtgatttgcaggtggacacgcactggcactaaatccacactgcccatctcggagacaggcggaggaactcccgagctgagatgcggaaagcaccaggccgtagggacctcaccgcagcgggccgcagctcaggactcggagcaggagagtgctgggatggaaggccgggatactcacaggctgccgagagccctctgagggcaaagtggcaggacactgcagaacagcttcatgttcccaaaatcaccgtacgactcttttccaaacacaggcaaatccgaaatcagcaggacgactgttttcccaacacgggtaaatctagtaccatgtcgtagttactctcaggcatggatgaataaatgttatagctgcatttgtctggccttttcttgtaaacattgcctgatttgttccttctggggctttgccattaaaatgtatttacagattaaaaaaa

SEQ ID NO: 24 - DNA, 470 bp, Homo sapiens
atgctttatcagaagatctgctgagcatcattgcaaacatgtctggatgtctcccttacatgctgcccccaaaatgcccaaacacttgcctggcgaacaaatacaggcccatcacaggagcttgcaacaacagagaccaccccagatggggcgcctccaacacggccctggcacgatggctccctccagtctatgaggacggcttcagtcagccccgaggctggaaccccggcttcttgtacaacgggttcccactgcccccggtccgggaggtgacaagacatgtcattcaagtttcaaatgaggttgtcacagatgatgaccgctattctgacctcctgatggcatggggacaatacatcgaccacgacatcgcgttcacaccacagagcaccagcaaagctgccttcgggggaggggctgactgccagatgacttgtgagaaccaaaacccatgttttcccatacaa

(19) United States
(12) Patent Application Publication
(10) Pub. No.: US 2023/0323465 A1
(43) Pub. Date: Oct. 12, 2023

(54) THYROID CANCER-SPECIFIC BIOMARKER PANEL

(71) Applicant: The Johns Hopkins University, Baltimore, MD (US)

(72) Inventors: Christopher B. Umbricht, Towson, MD (US); Yongchun Wang, Towson, MD (US); Martha A. Zeiger, Bethesda, MD (US)

(21) Appl. No.: 18/002,874

(22) PCT Filed: Jun. 25, 2021
(86) PCT No.: PCT/US2021/039043

§ 371 Date: Dec. 22, 2022

Related U.S. Application Data
(60) Provisional application No. 63/044,003, filed on Jun. 25, 2020.

(51) Int. Cl. C12Q 1/6851; C12Q 1/6886

(52) U.S. Cl. CPC C12Q 1/6886; C12Q 1/6851; C12Q 2600/156; C12Q 2600/158

(57) ABSTRACT

The present invention relates to the field of cancer. More specifically, the present invention provides compositions and methods directed to a thyroid cancer-specific biomarker panel. In a specific embodiment, a methods for identifying at thyroid tumor/nodule as benign or malignant comprises the steps of (a) measuring expression, in a sample obtained from the patient, by real-time quantitative polymerase chain reaction (RT-PCR) of at least three splice variant markers of a panel comprising high mobility group AT-hook 2 (HMGA2), transcript variant 1; PLAG1 zine finger (PLAG1), transcript variant 1; kallikrein related peptidase 7 (KLK7), transcript variant 1; fibronectin type III domain containing 4 (FNDC4); and cadherin 3 (CDH3), transcript variant 1; and (b) identifying the tumor as benign or malignant based on the measured expression levels of the panel of splice variant markers as compared to a control.

### CROSS-REFERENCE TO RELATED APPLICATIONS

This application claims the benefit of U.S. Provisional Application No. 63/044,003, filed Jun. 25, 2020, which is incorporated herein by reference in its entirety.

### FIELD OF THE INVENTION

The present invention relates to the field of cancer. More specifically, the present invention provides compositions and methods directed to a thyroid cancer-specific biomarker panel.

### INCORPORATION-BY-REFERENCE OF MATERIAL SUBMITTED ELECTRONICALLY

This application contains a sequence listing. It has been submitted electronically via EFS-Web as an ASCII text file entitled “P15313-02_ST25.txt.” The sequence listing is 36,157 bytes in size, and was created on Jun. 23, 2021. It is hereby incorporated by reference in its entirety.

### BACKGROUND OF THE INVENTION

Thyroid cancer incidence is rapidly increasing worldwide. In the United States, its prevalence has nearly quadrupled over the past 15 years, predominantly due to the increased incidence of papillary thyroid carcinoma (PTC) (1). Thyroid nodules are detectable by ultrasound in over 50% of the adult population, and fine needle aspiration (FNA) cytopathology is the most accurate means of pre-operative diagnosis. However, up to 30% of the FNA samples result in indeterminate cytology: atypia of undetermined significance/follicular lesion of undetermined significance (Bethesda III), follicular neoplasm/suspicious for a follicular neoplasm (Bethesda IV), and suspicious for malignancy (Bethesda V) (2), demonstrating inherent limitations of visual microscopic diagnosis. The situation has been further complicated by the recent reclassification in the American Thyroid Association management guidelines for thyroid tumors of a previously considered malignant subgroup of follicular variant of papillary thyroid carcinoma (FVPTC) to the noninvasive follicular thyroid neoplasm with papillary-like nuclear features (NIFTP), an indolent neoplasm with questionable malignant potential (3). NIFTP cannot be differentiated from invasive FVPTC by cytopathology, however, and requires histopathologic evaluation for diagnosis (2,4).

In the last decade, molecular testing has emerged as a promising strategy for the preoperative assessment of thyroid nodules to enable clinicians to better tailor surgical interventions and avoid overtreatment of benign disease. ThyroSeq v3 next generation sequencing (CBL Path, Rye Brook, NY) (5) Afirma gene expression with recently upgraded gene sequencing classifiers (Veracyte, South San Francisco, CA)(6), and ThygenX/ThyraMIR gene mutation and miRNA analysis (Interpace Diagnostics, Parsippany, NJ) (7) are major molecular tests currently in clinical use in the United States. The DNA-based tests, however, are limited by the prevalence of several of the assessed mutations in benign thyroid lesions (8,9), and all tests suffer from a lack of specificity after the introduction of the newly defined NIFTP subtype (10-13). Indeed, both ThyroSeq and Afirma tests currently report NIFTP as “positive” or “suspicious” for malignant disease. In a recent study, the positive predictive value (PPV) of ThyroSeq mutational analysis panels (including 7-gene ThyroSeq and ThyroSeq V2) decreased from 43% to 14% and Afirma gene expression classifier from 30% to 25% (14), if NIFTP was classified as non-malignant.

While molecular testing is a promising strategy for preoperative assessment of thyroid nodules, it further presents unique challenges for molecular assays including contaminating peripheral blood mononuclear cells (PBMC) and variable numbers of evaluable epithelial thyroid cells. Moreover, the newly recognized entity, noninvasive follicular thyroid neoplasm with papillary-like nuclear features (NIFTP), has added an additional challenge to the currently available molecular diagnostic platforms. New diagnostic tools are still needed to correctly distinguish benign and malignant thyroid nodules preoperatively.

### SUMMARY OF THE INVENTION

As described herein, the present inventors have improved on the prior art by characterizing the differential expression profile of specific splice variants, optimizing the ability of the molecular test for discriminating benign from malignant tumors, and also for classifying the newly introduced NIFTP subset of tumors. In particular embodiments, the present invention provides a quantitative RT-PCR based splice-variant-specific diagnostic gene panel optimized for evaluation of preoperative thyroid fine needle aspiration (FNA) cytology material, with high sensitivity and specificity to distinguish benign from malignant tumors.

The present invention also provides value in the stratification of special high risk populations or microcarcinomas who would benefit from active surveillance management over surgery. Such populations include patients with very low risk tumors (e.g., papillary microcarcinomas without clinically evident metastases or local invasion, and no convincing cytologic evidence of aggressive disease); patients at high surgical risk because of comorbid conditions; patients expected to have a relatively short remaining life span (e.g., serious cardiopulmonary disease, other malignancies, very advanced age); and patients with concurrent medical or surgical issues that need to be addressed prior to thyroid surgery.

In one aspect, the present invention provides compositions and methods useful for identifying at thyroid tumor/nodule as benign or malignant. In one embodiment, the method comprises the steps of (a) measuring expression, in a sample obtained from the patient, by real-time quantitative polymerase chain reaction (RT-qPCR) of at least three splice variant markers of a panel comprising high mobility group AT-hook 2 (HMGA2), transcript variant 1 (NCBI Reference Sequence, NM_003483.5); PLAG1 zine finger (PLAG1), transcript variant 1 (NCBI Reference Sequence, NM_002655.3); kallikrein related peptidase 7 (KLK7), transcript variant 1 (NCBI Reference Sequence, NM_005046.4); fibronectin type III domain containing 4 (FNDC4) (NCBI Reference Sequence, NM_022823.3); and cadherin 3 (CDH3), transcript variant 1 (NCBI Reference Sequence NM_001793.6); and (b) identifying the tumor as benign or malignant based on the measured expression levels of the panel of splice variant markers as compared to a control.

In particular embodiments, the sample is a fine needle aspiration (FNA) biopsy. In certain embodiments, the markers in the panel are not detectable in peripheral blood mononuclear cells. In some embodiments, the method further comprises detecting the presence of thyroid epithelial cells in the sample. In a specific embodiment, the detecting step comprises measuring the expression of thyroid peroxidase isoform 1 (TPO1). In a more specific embodiment, RT-qPCR is performed using primers that amplify all or a part of nucleotides 441-910 of SEQ ID NO:23 (TPO1). In an even more specific embodiment, the primers comprise at least one of SEQ ID NOS: 11-12.

In certain embodiments, RT-qPCR is performed using primers that amplify all or a part of the following regions of the markers: nucleotides 961-1320 of SEQ ID NO: 13 (HMGA2); nucleotides 154-513 of SEQ ID NO: 15 (PLAG1); nucleotides 204-563 of SEQ ID NO: 17 (KLK7); nucleotides 481-800 of SEQ ID NO:19 (FNDC4); and nucleotides 767-1246 of SEQ ID NO:21 (CDH3). In a specific embodiment, the HMGA2 primers comprise at least one of SEQ ID NOS: 1-2. In another specific embodiment, the PLAG1 primers comprise at least one of SEQ ID NOS:3-4. In a further embodiment, the KLK7 primers comprise at least one of SEQ ID NOS:5-6. In yet another embodiment, the FNDC4 primers comprise at least one of SEQ ID NOS:7-8. In another embodiment, the CDH3 primers comprise at least one of SEQ ID NOS:9-10.

In particular embodiment, the patient sample is from a thyroid FNA previously determined to be indeterminate. The preset invention can also be used to distinguish between non-invasive follicular thyroid neoplasm with papillary-like nuclear features (NIFTP) from malignant follicular variant of papillary thyroid cancer (FVPTC).

In certain embodiments, the identifying step comprises normalizing marker expression to TPO1; z-transforming to create a composite score; and performing a receiver operating characteristic (ROC) analysis.

In particular embodiments, the method comprises extracting total RNA from the sample and reverse transcribing total RNA. The method can further comprise performing RT-PCR to detect the presence of thyroid epithelial cells in the sample. In one embodiment, the detecting step comprises measuring expression of a thyrocyte-specific marker. In further embodiments, the method comprises performing qPCR of markers that are undetectable in white blood cells, which are unavoidable in FNA derived materials.

In particular embodiments, the present invention has at least an 85% ability to distinguish between thyroid malignant and benign tumor, with a specificity of at least 90% and a sensitivity of at least 75%, and a combined negative predictive value of at least 90% and a positive predictive value of at least 70%. In specific embodiments, the present invention has at least a 75% ability to differentiate indolent NIFTP from invasive FVPT and PTC with achieved specificity of at least 85% and sensitivity of at least 60%.

The present invention further comprises treating a patient who is identified as having a malignant tumor with a thyroidectomy, hemithyroidectomy, radioactive iodine therapy, and combinations thereof. In one embodiment, treatment further comprises one or more of a TERT inhibitor, a BRAF V600E inhibitor, a MEK inhibitor or combinations thereof. In other embodiments, the treatment comprises an anti-cancer drug.

In another aspect, the present invention provides compositions and methods for treating a patient having a malignant thyroid tumor or nodule. In particular embodiments, a method comprises the step of performing one or more of a thyroidectomy, hemithyroidectomy, and radioactive iodine therapy and/or administering one or more of a TERT inhibitor, a BRAF V600E inhibitor, and a MEK inhibitor to a patient identified as having a malignant thyroid tumor based on expression of at least three of the following splice variant markers: HMGA2, transcript variant 1 (NCBI Reference Sequence, NM_003483.5); PLAG1, transcript variant 1 (NCBI Reference Sequence, NM_002655.3); KLK7, transcript variant 1 (NCBI Reference Sequence, NM_005046.4); FNDC4 (NCBI Reference Sequence, NM_022823.3); and CDH3, transcript variant 1 (NCBI Reference Sequence NM _001793.6).

In another embodiment, a method for treating a patient having a malignant thyroid tumor or nodule comprises the steps of (a) measuring expression, in a sample obtained from the patient, by RT-qPCR of at least three splice variant markers of a panel comprising HMGA2, transcript variant 1 (NCBI Reference Sequence, NM_003483.5); PLAG1, transcript variant 1 (NCBI Reference Sequence, NM_002655.3); KLK7, transcript variant 1 (NCBI Reference Sequence, NM_005046.4); FNDC4 (NCBI Reference Sequence, NM_022823.3); and CDH3, transcript variant 1 (NCBI Reference Sequence NM_001793.6); (b) identifying the tumor as malignant based on the measured expression levels of the panel of splice variant markers as compared to a control; and (c) treating the patient with one or more of a thyroidectomy, hemithyroidectomy, radioactive iodine therapy, TERT inhibitor, BRAF V600E inhibitor, and MEK inhibitor.

### BRIEF DESCRIPTION OF THE FIGURES

FIGS. 1A-1B. Expression of the 5 gene transcripts and internal controls (TPO1 & GapDH) in solid thyroid tumor frozen samples. FIG. 1A: Representative gel image showing each of the 5 isoforms and thyrocyte-specific TPO1 in 8 thyroid tumor subtypes. FIG. 1B: Bar plot of the sum of expression levels of the 5 isoforms (AN, n = 15; FA, n = 14; HA, n = 10; NIFTP, n = 5; FVPTC, n = 7; HC, n = 7; FC, n = 7; PTC, n = 15). Semi-quantitative densitometry of each isoform was normalized to GapDH and z-transformed so that all genes would be on the same scale.

FIGS. 2A-2F. The expression profiles of the 5 gene expression isoforms in FNAs. The box plots show the 5^{th}, 25^{th}, 50^{th}, 75^{th} and 95^{th} percentiles of the sample expression z-ΔCt scores in each thyroid tumor subgroup. FIG. 2F shows the sum of 5-transcript composite z-ΔCt score. The expression reference line is the z-ΔCt score of -1, the threshold corresponding to the sensitivity of 75% at the specificity of 91%. AN, n = 25; FA, n = 16; HA, n = 14; NIFTP, n = 23; FVPTC, n = 34; PTC, n = 25.

FIGS. 3A-3C. ROC Curves of the diagnostic power of the 5-transcript panel in FNAs. FIG. 3A: Benign (AN, FA, HA, and NIFTP, n = 78) vs. malignant (FVPTC and PTC, n = 59) thyroid tumors. The dot on the ROC curve is the threshold for the composite expression z-ΔCt score of -1, corresponding to the sensitivity (75%) at specificity 91%. FIG. 3B: Thyroid follicular neoplasms, malignant FVPTC (n = 34) vs. FA (n = 16) and NIFTP (n = 23). FIG. 3C: NIFTP (n = 23) vs. malignant thyroid tumors, FVPTC and PTC (n = 59).

FIG. 4. Study Workflow Diagram.

FIG. 5. Expression of the 5 gene transcripts and internal control in PBMC samples. Lane 1-5: PBMCs from different patients. Center lane: 50-bp DNA ladder. The representative gel image shows that in contrast to the general house-keeping gene GapDH, none of the 5 isoforms were detectable in patient PBMCs.

FIG. 6. The expression of different TPO1 and Thyroglobulin isoforms in PBMC samples. The representative gel image shows TPO1 was the only isoform not detectable in PBMCs.

FIG. 7. Semiquantitative PCR on thyroid solid tumors. PCR products were quantified using Quantity One image analysis software (version 4.6.0; BioRad). AN, adenomatoid nodule, n=10; FA, follicular adenoma, n=9; EFC, encapsulated FBPTC=NIFTP, n=1-; FV, FVPTC with known invasion, n=6; PTC, papillary thyroid carcinoma.

FIG. 8. Semiquantitative PCR on thyroid solid tumors, 3 gene model. PCR products were quantified using Quantity One image analysis software (version 4.6.0; BioRad). AN, adenomatoid nodule, n=10; FA, follicular adenoma, n=9; EFV, encapsulated follicular variant of papillary thyroid carcinoma, n=10; FV, follicular variant of papillary thyroid carcinoma with known invasion, n=6; PTC, papillary thyroid carcinoma, n=9.

FIG. 9. qPCR on FNA samples, reference TPO1.

FIG. 10. qPCR test on FNA samples. Genes normalized to TPO 1 using 2^{(ΔcT)}method.

FIG. 11. qPCR test on FNA samples. Genes normalized to TPO1 using 2^{(ΔcT)} method.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The present invention can be used to assess indeterminate thyroid FNA to establish risk/presence of cancer. Indeed, current molecular tests assessing thyroid nodules that cannot be assessed through cytology suffer from lack of specificity and high false positive rates, resulting in unnecessary surgery. In certain embodiments, the present invention provides a RT-PCR based assay with the ability to distinguish benign from malignant thyroid nodules with high specificity and/or distinguish indolent (slow growing) from metastatic (aggressive) cancers. The present invention can reduce unnecessary surgical thyroid procedures for benign cases and/or inform on appropriate surgical procedure or monitoring for indolent versus metastatic cases.

As described herein, twenty-two transcript splice variants from 12 genes we previously identified as discriminating benign from malignant thyroid nodules were characterized in 80 frozen thyroid tumors from 8 histological subtypes. Isoforms detectable in PBMC were excluded, and the 5 most discriminating isoforms were further validated by real-time quantitative PCR (qPCR) on intraoperative FNA samples from 59 malignant tumors, 55 benign nodules, and 23 NIFTP samples. The qPCR threshold cycle values for each transcript were normalized to the thyrocyte-specific thyroid peroxidase isoform 1 (TPO1) and z-transformed. Receiver operating characteristic (ROC) analyses of the composite transcript scores were used to evaluate classification of thyroid FNAs by the 5-gene isoform expression panel.

In particular embodiments, a molecular signature was developed by combining expression levels of specific isoforms of CDH3, FNDC4, HMGA2, KLK7, and PLAG1. FNAs containing at least 12-36 thyrocytes were sufficient for this assay. The 5-gene composite score achieved an area under the ROC curve (AUC) of 0.86 for distinguishing malignant from benign nodules, with a specificity of 91%, sensitivity of 75%, negative predictive value of 91% and positive predictive value of 74%. This 5-gene isoform expression panel embodiment distinguishes benign from malignant thyroid tumors and, may help distinguish benign from malignant thyroid nodules in the context of the new NIFTP subtype.

### I. Definitions

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%. An alteration may be, for example, a change in expression level or activity.

By “control” is meant a standard or reference condition. For example, gene expression in a sample from a patient having thyroid cancer may be compared to the level of gene expression from the same patient at an earlier time, from a patient not having thyroid cancer, and the like.

In certain embodiments, a reference or control can be from normal thyroid tissue, cancerous thyroid tissue or any other type of thyroid tissue for which a classification is known. As used herein, “a cell of a normal subject” or “normal thyroid tissue” means a cell or tissue which is histologically normal and was obtained from a subject believed to be without malignancy and having no increased risk of developing a malignancy or was obtained from tissues adjacent to tissue known to be malignant and which is determined to be histologically normal (non-malignant) as determined by a pathologist. The reference cell population can be from any subject, including cells of the subject being tested obtained prior to developing the condition that lead to the testing. The normal reference cell population can be homogeneous for normal cells.

“Detect” refers to identifying the presence, absence or amount of the analyte to be detected. In certain embodiments, “detect” is used interchangeably with “measure.”

The term “nucleotide sequence” refers to a polymer of DNA or RNA which can be single-stranded or double-stranded, optionally containing synthetic, non-natural or altered nucleotide bases capable of incorporation into DNA or RNA polymers. A DNA molecule or polynucleotide is a polymer of deoxyribonucleotides (A, G, C, and T), and an RNA molecule or polynucleotide is a polymer of ribonucleotides (A, G, C and U).

The terms “complementary” and “complementarity” refer to polynucleotides (i.e., a sequence of nucleotides) related by the base-pairing rules. For example, the sequence “A-G-T”, is complementary to the sequence “T-C-A.” Complementarity may be “partial”, in which only some of the nucleic acids’ bases are matched according to the base pairing rules. Alternatively, there may be “complete” or “total” complementarity between the nucleic acids. The degree of complementarity between nucleic acid strands has significant effects on the efficiency and strength of hybridization between nucleic acid strands.

A “gene,” for the purposes of the present invention, includes a DNA region encoding a gene product, as well as all DNA regions which regulate the production of the gene product, whether or not such regulatory sequences are adjacent to coding and/or transcribed sequences. The term “gene” is used broadly to refer to any segment of nucleic acid associated with a biological function. Genes include coding sequences and/or the regulatory sequences required for their expression. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. For example, “gene” refers to a nucleic acid fragment that expresses mRNA, functional RNA, or specific protein, including regulatory sequences. “Functional RNA” refers to sense RNA, antisense RNA, ribozyme RNA, siRNA, or other RNA that may not be translated but yet has an effect on at least one cellular process. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

“Gene expression” refers to the conversion of the information contained in a gene into a gene product. It refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein. The term “altered level of expression” refers to the level of expression in cells or organisms that differs from that of normal cells or organisms.

A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. The term “RNA transcript” refers to the product resulting from RNA polymerase catalyzed transcription of a DNA sequence. When the RNA transcript is a perfect, complementary copy of the DNA sequence, it is referred to as the primary transcript or it may be a RNA sequence derived from post-transcriptional processing of the primary transcript and is referred to as the mature RNA “Messenger RNA” (mRNA) refers to the RNA that is without intrans and that can be translated into protein by the cell. “cDNA” refers to a single- or a double-stranded DNA that is complementary to and derived from mRNA. “Functional RNA” refers to sense RNA, antisense RNA, ribozyme RNA, siRNA, or other RNA that may not be translated but yet has an effect on at least one cellular process.

A “coding sequence,” or a sequence that “encodes” a selected polypeptide, is a nucleic acid molecule that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral (e.g., DNA viruses and retroviruses) or prokaryotic DNA, and especially synthetic DNA sequences. A transcription termination sequence may be located 3′ to the coding sequence.

Certain embodiments of the disclosure encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The term “expression” refers to the transcription and/or translation and/or activity of a gene described herein. Several methods can be utilized to determine the level of expression, as described in detail below.

The term “sample” as used herein refers to any biological specimen that may be extracted, untreated, treated, diluted or concentrated from a subject. The sample can be a biological sample. The biological samples are generally derived from a patient, including a cell sample or bodily fluid (such as tumor tissue, lymph node, sputum, blood, bone marrow, cerebrospinal fluid, phlegm, saliva, or urine) or cell lysate. The cell lysate can be prepared from a tissue sample (e.g., a tissue sample obtained by biopsy), for example, a tissue sample (e.g., a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In specific embodiments, the sample is a stool sample. In other embodiments, the sample is blood. In particular embodiments, the sample is a fine needle aspiration (FNA) sample.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

“Protein,” “polypeptide” and “peptide” are used interchangeably herein to refer to a polymer of amino acid residues and to variants and synthetic analogues of the same.

The term “antibody” and its grammatical equivalents refer to a protein which is capable of specifically binding to a target antigen and includes any substance, or group of substances, which has a specific binding affinity for an antigen, suitably to the exclusion of other substances. This term encompasses an immunoglobulin molecule capable of specifically binding to a target antigen by virtue of an antigen binding site contained within at least one variable region. This term includes four chain antibodies (e.g., two light chains and two heavy chains), recombinant or modified antibodies (e.g., chimeric antibodies, humanized antibodies, primatized antibodies, de-immunized antibodies, half antibodies, bispecific antibodies) and single domain antibodies such as domain antibodies and heavy chain only antibodies (e.g., camelid antibodies or cartilaginous fish immunoglobulin new antigen receptors (IgNARs)).

In one example, the antibody is a murine (mouse or rat) antibody or a primate (suitably human) antibody. The term “antibody” encompasses not only intact polyclonal or monoclonal antibodies, but also variants, fusion proteins comprising an antibody portion with an antigen binding site, humanized antibodies, human antibodies, chimeric antibodies, primatized antibodies, de-immunized antibodies or veneered antibodies. Also within the scope of the term “antibody” are antigen-binding fragments that retain specific binding affinity for an antigen, suitably to the exclusion of other substances. This term includes a Fab fragment, a Fab′ fragment, a F(ab′) fragment, a single chain antibody, and the like.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

The terms “marker” or “biomarker” are used interchangeably, broadly refer to any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a sample. “Derived from” as used in this context refers to a compound that, when detected, is indicative of a particular molecule being present in the sample. For example, detection of a particular cDNA can be indicative of the presence of a particular RNA transcript in the sample. As another example, detection of or binding to a particular antibody can be indicative of the presence of a particular antigen (e.g., protein) in the sample. Here, a discriminating molecule or fragment is a molecule or fragment that, when detected, indicates presence or abundance of an above-identified compound. A biomarker can, for example, be isolated from a sample, directly measured in a sample, or detected in or determined to be in a sample. A biomarker can, for example, be functional, partially functional, or non-functional.

The “level”, “abundance” or “amount” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment. In specific embodiments in which the level of a biomarker is “reduced” relative to a reference or control, the reduced level may refer to an overall reduction of any of at least about 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the decrease is at least about any of at least 0.9x, 0.8x, 0.7x, 0.6x, 0.5x, 0.4x, 0.3x, 0.2x, 0.1, 0.05x, 0.01x, and the like, the level/amount of the respective biomarker in a reference sample, reference cell, reference tissue, control sample, control cell, or control tissue. In specific embodiments in which the level of a biomarker is “increased” relative to a reference or control, the reduced level may refer to an overall increase of any of at least about 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the increase is at least about any of at least about 1.1x, 1.2x, 1.3x, 1.4x, 1.5x, 1.6x, 1.7x, 1.8x, 1.9x, 2x, 3x, 4x, 5x, 10x, 20x, 30x, 50x, 80x, 100x, and the like, the level/amount of the respective biomarker in a reference sample, reference cell, reference tissue, control sample, control cell, or control tissue. In certain embodiments in which the level of a biomarker is “about the same” a reference or control, the level of biomarker varies by less than about 10%, 9%, 8%, 7%, 6%, 5%, 4%, 3%, 2%, 1%, 0.5%, 0.1%, or even less, as compared to the level of biomarker (e.g., protein or nucleic acid (e.g., mRNA or cDNA)), detected by standard art known methods such as those described herein, in a reference sample, reference cell, reference tissue, control sample, control cell, or control tissue.

The term “indicator” as used herein refers to a result or representation of a result, including any information, number, ratio, signal, sign, mark, or note by which a skilled artisan can estimate and/or determine a likelihood of whether or not a subject has a condition or a subject suffering from the condition will respond to a relevant therapy. An “indicator” may optionally be used together with other clinical characteristics to arrive at a determination that the subject has a condition or is or is not likely to respond to a therapy. That such an indicator is “determined” is not meant to imply that the indicator is 100% accurate. The skilled clinician may use the indicator together with other clinical indicia to arrive at a conclusion.

As used herein, the term “label” and grammatical equivalents thereof, refer to any atom or molecule that can be used to provide a detectable and/or quantifiable signal. In particular, the label can be attached, directly or indirectly, to a nucleic acid or protein. Suitable labels that can be attached include, but are not limited to, radioisotopes, fluorophores, quenchers, chromophores, mass labels, electron dense particles, magnetic particles, spin labels, molecules that emit chemiluminescence, electrochemically active molecules, enzymes, cofactors, and enzyme substrates. A label can include an atom or molecule capable of producing a visually detectable signal when reacted with an enzyme. In some embodiments, the label is a “direct” label which is capable of spontaneously producing a detectible signal without the addition of ancillary reagents and is detected by visual means without the aid of instruments. For example, colloidal gold particles can be used as the label. Many labels are well known to those skilled in the art. In specific embodiments, the label is other than a naturally-occurring nucleoside. The term “label” also refers to an agent that has been artificially added, linked or attached via chemical manipulation to a molecule.

As used herein, “primer,” “probe,” and “oligonucleotide” are used interchangeably.

The term “nucleic acid probe” or a “probe specific for” a nucleic acid refers to a nucleic acid sequence that has at least about 80%, e.g., at least about 90%, e.g., at least about 95%, 96%, 97%, 98%, 99% contiguous sequence identity or homology to the nucleic acid sequence encoding the targeted sequence of interest. A probe (or oligonucleotide or primer) of the disclosure is at least about 8 nucleotides in length (e.g., at least about 8-50 nucleotides in length, e.g., at least about 10-40, e.g., at least about 15-35 nucleotides in length). The oligonucleotide probes or primers of the disclosure may comprise at least about eight nucleotides at the 3′ of the oligonucleotide that have at least about 80%, e.g., at least about 85%, e.g., at least about 90% contiguous identity to the targeted sequence of interest.

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded; if double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including application, temperature to be employed, template reaction conditions, other reagents, and source of primers. The oligonucleotide primer typically contains 12-20 or more nucleotides, although it may contain fewer nucleotides. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide.

In certain embodiments, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

As used herein, the term “probe” refers to a molecule that binds to a specific sequence or sub-sequence or other moiety of another molecule. Unless otherwise indicated, the term “probe” typically refers to a nucleic acid probe that binds to another nucleic acid, also referred to herein as a “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physicochemical interactions typically occur in interactions between molecules. In particular embodiments, all that is required is that the molecules (e.g., nucleic acids or polypeptides) remain immobilized or attached to a support under conditions in which it is intended to use the support, for example in applications requiring nucleic acid amplification and/or sequencing or in in antibody-binding assays. For example, oligonucleotides or primers are immobilized such that a 3′ end is available for enzymatic extension and/or at least a portion of the sequence is capable of hybridizing to a complementary sequence. In some embodiments, immobilization can occur via hybridization to a surface attached primer, in which case the immobilized primer or oligonucleotide may be in the 3′-5′ orientation. In other embodiments, immobilization can occur by means other than base-pairing hybridization, such as the covalent attachment.

The term “solid support” as used herein refers to a solid inert surface or body to which a molecular species, such as a nucleic acid and polypeptides can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

As used herein, the terms “diagnosis,” “diagnosing” and the like are used interchangeably herein to encompass determining the likelihood that a subject will develop or has a condition or clinical state. These terms also encompass, for example, determining the level of clinical state (e.g., the level of responsiveness to a therapy), as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or derived biomarker values actually has a condition or clinical state (or not) based on a given mathematical model. An increased likelihood for example may be relative or absolute and may be expressed qualitatively or quantitatively. For instance, an increased likelihood may be determined simply by determining the subject’s measured biomarker levels and placing the subject in an “increased likelihood” category, based upon previous population studies. The term “likelihood” is also used interchangeably herein with the term “probability”.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, the term “positive response” means that the result of a treatment regimen includes some clinically significant benefit, such as the prevention, or reduction of severity, of symptoms, or a slowing of the progression of the condition. By contrast, the term “negative response” or “non-response” means that a treatment regimen provides no or minimal clinically significant benefit, such as the prevention, or reduction of severity, of symptoms, or increases the rate of progression of the condition.

The term “prognosis” as used herein refers to a prediction of the probable course and outcome of a clinical condition or disease. A prognosis is usually made by evaluating factors or symptoms of a disease or condition that are indicative of a favorable or unfavorable course or outcome of the disease or condition (e.g., response to therapy). The skilled artisan will understand that the term “prognosis” refers to an increased probability that a certain course or outcome will occur; that is, that a course or outcome is more likely to occur in a subject exhibiting a given condition, when compared to those individuals not exhibiting the condition.

The term “classifying a thyroid lesion” is equivalent to diagnosing a subject with a type of thyroid lesion. These lesions can be benign or malignant. Examples of a benign lesion include, but are not limited to, follicular adenoma, hyperplastic nodule, papillary adenoma, thyroiditis nodule, multinodular goiter, adenomatoid nodules, Hurthle cell adenomas, and lymphocytic thyroiditis nodules. Examples of malignant lesions include, but are not limited to, papillary thyroid carcinoma, follicular variant of papillary thyroid carcinoma, follicular carcinoma, Hurthle cell tumor, anaplastic thyroid cancer, medullary thyroid cancer, thyroid lymphoma, poorly differentiated thyroid cancer and thyroid angiosarcoma.

Once a subject has been diagnosed with a malignant lesion or thyroid tumor, the stage of thyroid malignancy can also be determined by the methods of the present invention. Staging of a thyroid malignancy or tumor can be useful in prescribing treatment as well as in determining a prognosis for the subject.

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

Optional″ or “optionally” means that the subsequently described event or circumstance can or cannot occur, and that the description includes instances where the event or circumstance occurs and instances where it does not.

It will be appreciated that the terms used herein and associated definitions are used for the purpose of explanation only and are not intended to be limiting.

### II. Sample Preparation for Detection of Expression

In certain embodiments, a sample from a subject with or suspected of having thyroid cancer is processed prior to detection or quantification. For example, nucleic acid and/or proteins may be extracted, isolated, and/or purified from a sample prior to analysis. Various DNA, mRNA, and/or protein extraction techniques are well known to those skilled in the art. Processing may include centrifugation, ultracentrifugation, ethanol precipitation, filtration, fractionation, resuspension, dilution, concentration, etc. In some embodiments, methods and systems provide detection (e.g., quantification of RNA or protein biomarkers) from raw sample (e.g., biological fluid such as blood, serum, etc.) without or with limited processing. In some examples, whole cells or tissue sections are isolated and analyzed for expression such as using immunohistochemistry (IHC) or flow cytometry.

Methods may comprise steps of homogenizing a sample in a suitable buffer, removal of contaminants and/or assay inhibitors, adding a biomarker capture reagent (e.g., a magnetic bead to which is linked an oligonucleotide complementary to a biomarker), incubated under conditions that promote the association (e.g., by hybridization) of the target biomarker with the capture reagent to produce a target biomarker: capture reagent complex, incubating the target biomarker: capture complex under target biomarker-release conditions. In some embodiments, multiple biomarkers are isolated in each round of isolation by adding multiple biomarkers capture reagents (e.g., specific to the desired biomarkers) to the solution. For example, multiple biomarker capture reagents, each comprising an oligonucleotide specific for a different biomarker can be added to the sample for isolation/detection/measurement of multiple biomarkers. It is contemplated that the methods encompass multiple experimental designs that vary both in the number of capture steps and in the number of target biomarkers captured in each capture step.

In some embodiments, capture reagents are molecules, moieties, substances, or compositions that preferentially (e.g., specifically and selectively) interact with a particular biomarker sought to be isolated, purified, detected, and/or quantified. Any capture reagent having desired binding affinity and/or specificity to the particular biomarker can be used in the present technology.

For example, the capture reagent can be a macromolecule such as a peptide, a protein (e.g., an antibody or other ligand that specifically binds to the biomarker), an oligonucleotide, a nucleic acid (e.g., nucleic acids capable of hybridizing with the biomarker gene), oligosaccharides, carbohydrates, lipids, or small molecules, or a complex thereof. As illustrative and non-limiting examples, an avidin target capture reagent may be used to isolate and purify targets comprising a biotin moiety, an antibody may be used to isolate and purify targets comprising the appropriate antigen or epitope, and an oligonucleotide may be used to isolate and purify a complementary polynucleotide.

Any nucleic acids, including single-stranded and double-stranded nucleic acids, which are capable of binding, or specifically binding, to a target biomarker can be used as the capture reagent. Examples of such nucleic acids include DNA, RNA, aptamers, peptide nucleic acids, and other modifications to the sugar, phosphate, or nucleoside base. Thus, there are many strategies for capturing a target and accordingly many types of capture reagents are known to those in the art.

In addition, target biomarker capture reagents may comprise a functionality to localize, concentrate, aggregate, etc., the capture reagent and thus provide a way to isolate and purify the target biomarker when captured (e.g., bound, hybridized, etc.) to the capture reagent (e.g., when a target: capture reagent complex is formed). For example, in some embodiments the portion of the capture reagent that interacts with the biomarker (e.g., an oligonucleotide) is linked to a solid support (e.g., a bead, surface, resin, column, and the like) that allows manipulation by the user on a macroscopic scale. Often, the solid support allows the use of a mechanical means to isolate and purify the target: capture reagent complex from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target biomarker) from the heterogeneous solution.

The target biomarker may be quantified or detected using any suitable technique. In specific embodiments, the biomarker is quantified using reagents that determine the level, abundance or amount of the individual biomarker, either as isolated biomarker or as expressed in or on a cell. Non-limiting reagents of this type include reagents for use in nucleic acid- and protein-based assays, as described below.

### III. Detection of Target Nucleic Acid

Many methods of measuring the levels or amounts of biomarker nucleic acid expression are contemplated. Any reliable, sensitive, and specific method can be used. In particular embodiments, biomarker nucleic acid is amplified prior to measurement. In other embodiments, the level of biomarker nucleic acid is measured during the amplification process. In still other methods, the target nucleic acid is not amplified prior to measurement.

### A. Amplification Reactions

Many methods exist for amplifying nucleic acid sequences. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR).

A typical PCR reaction comprises multiple amplification steps or cycles that selectively amplify target nucleic acid species including a denaturing step in which a target nucleic acid is denatured; an annealing step in which a set of PCR primers (forward and reverse primers) anneal to complementary DNA strands; and an extension step in which a thermostable DNA polymerase extends the primers. By repeating these steps multiple times, a DNA fragment is amplified to produce an amplicon, corresponding to the target DNA sequence. Typical PCR reactions include about 20 or more cycles of denaturation, annealing, and extension. In many cases, the annealing and extension steps can be performed concurrently, in which case the cycle contains only two steps. Because mature mRNA are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than about 15, fewer than about 20, fewer than about 25, fewer than about 30, or fewer than about 35 nucleotides in length. In additional embodiments, a primer is at least about 35 nucleotides in length.

In a further embodiment, a forward primer can comprise at least one sequence that anneals to biomarker nucleic acid sequence and alternatively can comprise an additional 5′ non-complementary region. In another embodiment, a reverse primer can be designed to anneal to the complement of a reverse transcribed mRNA. The reverse primer may be independent of the biomarker nucleic acid sequence, and multiple biomarker nucleic acid sequences may be amplified using the same reverse primer. Alternatively, a reverse primer may be specific for a biomarker nucleic acid.

In some embodiments, two or more biomarker nucleic acid sequences are amplified in a single reaction volume. One aspect includes multiplex q-PCR, such as qRT-PCR, which enables simultaneous amplification and quantification of at least two biomarker nucleic acid sequences of interest in one reaction volume by using more than one pair of primers and/or more than one probe. The primer pairs comprise at least one amplification primer that uniquely binds each mRNA, and the probes are labeled such that they are distinguishable from one another, thus allowing simultaneous quantification of multiple biomarker nucleic acid sequences. Multiplex qRT-PCR has research and diagnostic uses including, but not limited, to detection of biomarker nucleic acid sequences for diagnostic, prognostic, and therapeutic applications.

The qRT-PCR reaction may further be combined with the reverse transcription reaction by including both a reverse transcriptase and a DNA-based thermostable DNA polymerase. When two polymerases are used, a “hot start” approach may be used to maximize assay performance. See U.S. Pat. Nos. 5,985,619 and No. 5,411,876. For example, the components for a reverse transcriptase reaction and a PCR reaction may be sequestered using one or more thermoactivation methods or chemical alteration to improve polymerization efficiency. See U.S. Pat. Nos. 6,403,341; No. 5,550,044; and No. 5,413,924.

### B. Detection Reactions

In certain embodiments, labels, dyes, or labeled probes and/or primers are used to detect amplified or unamplified biomarker nucleic acid sequence (mRNA/cDNA). One of ordinary skill in the art will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where biomarker nucleic acid sequence amplification is preferred.

A probe or primer may include Watson-Crick bases or modified bases. Modified bases include, but are not limited to, the AEGIS bases (from EraGen Biosciences, Inc. (Madison, WI)), which have been described, e.g., in U.S. Pat. Nos. 6,001,983; No. 5,965,364; and No. 5,432,272. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan®) probes (see U.S. Pat. Nos. 5,538,848), stem-loop molecular beacons (see, e.g., U.S. Pat. Nos. 6,103,476 and No. 5,925,517), stemless or linear beacons (see, e.g., WO 9921881, U.S. Pat. Nos. 6,649,349 and No. 6,485,901), peptide nucleic acid (PNA) Molecular Beacons (see, e.g., U.S. Pat. Nos. 6,593,091 and No. 6,355,421), linear PNA beacons (see, e.g., U.S. Pat. No. 6,329,144), non-FRET probes (see, e.g., U.S. Pat. Nos. 6,150,097), Sunrise®/Amplifluor® probes (see, e.g., U.S. Pat. Nos. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse® probe (Sigma-Aldrich Corp. (St. Louis, MO)), hairpin probes (see, e.g., U.S. Pat. No. 6,596,490), PNA light-up probes, antiprimer quench probes (Li et al., 53 CLIN. CHEM. 624-33 (2006)), self-assembled nanoparticle probes, and ferrocene-modified probes described, for example, in U.S. Patent No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Biomarker nucleic acid sequences can be detected by direct or indirect methods. In a direct detection method, one or more biomarker nucleic acid sequences are detected by a detectable label that is linked to a nucleic acid molecule. In such methods, the biomarker nucleic acid sequences may be labeled prior to binding to the probe. Therefore, binding is detected by screening for the labeled biomarker nucleic acid sequence that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified mRNA/cDNA, are detected using xMAP Microspheres (Luminex Corp. (Austin, TX)) conjugated with probes to capture the desired nucleic acids. Some methods may involve detection with polynucleotide probes modified, for example, with fluorescent labels or branched DNA (bDNA) detection.

In other embodiments, nucleic acids are detected by indirect detection methods. For example, a biotinylated probe may be combined with a stretavidin-conjugated dye to detect the bound nucleic acid. The streptavidin molecule binds a biotin label on amplified nucleic acid, and the bound nucleic acid is detected by detecting the dye molecule attached to the streptavidin molecule. In one embodiment, the streptavidin-conjugated dye molecule comprises Phycolink® Streptavidin R-Phycoerythrin (ProZyme, Inc. (Heward, CA)). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal. See, e.g., Garman A., Non-Radioactive Labeling, Academic Press (1997) and Kricka, L., Nonisotopic DNA Probe Techniques, Academic Press, San Diego (1992). Fluorescent reporter dyes useful as labels include, but are not limited to, fluoresceins (see, e.g., U.S. Pat. Nos. 6,020,481; No. 6,008,379; and No. 5,188,934), rhodamines (see, e.g., U.S. Pat. Nos. 6,191,278; No. 6,051,719; No. 5,936,087; No. 5,847,162; and No. 5,366,860), benzophenoxazines (see, e.g., U.S. Pat. No. 6,140,500), energy-transfer fluorescent dyes, comprising pairs of donors and acceptors (see, e.g., U.S. Pat. Nos. 5,945,526; No. 5,863,727; and 5,800,996; and), and cyanines (see, e.g., WO 9745539), lissamine, phycoerythrin, Cy2, Cy3, Cy3.5, Cy5, Cy5.5, Cy7, FluorX (Amersham Biosciences, Inc. (Piscataway, NJ)), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′, 1,4, - tetrachlorofluorescein, and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In further embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify biomarker nucleic acid may be used including, but not limited to, oligonucleotide ligation (OLA) methods and methods that allow a distinguishable probe that hybridizes to the target nucleic acid sequence to be separated from an unbound probe. For example, HARP-like probes, as disclosed in U.S. Pat. Application Publication No. 2006/0078894 may be used to measure the quantity of target nucleic acid. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

In an additional embodiment of the method, a probe ligation reaction may be used to quantify target biomarker nucleic acid. In a Multiplex Ligation-dependent Probe Amplification (MLPA) technique, pairs of probes which hybridize immediately adjacent to each other on the target nucleic acid are ligated to each other only in the presence of the target nucleic acid. See Schouten et al., 30 NUCL. ACIDS RES. e57 (2002). In some aspects, MLPA probes have flanking PCR primer binding sites. MLPA probes can only be amplified if they have been ligated, thus allowing for detection and quantification of biomarkers.

Furthermore, a sample may also be analyzed by means of a microarray. Microarrays generally comprise solid substrates and have a generally planar surface, to which a capture reagent (also called an adsorbent or affinity reagent) is attached. Frequently, the surface of a microarray comprises a plurality of addressable locations, each of which has the capture reagent (e.g., miRNA probes specific for particular biomarkers) bound there. Many microarrays are described in the art. These include, for example, biochips produced by Asuragen, Inc. (Austin, TX); Affymetrix, Inc. (Santa Clara, CA); GenoSensor Corp. (Tempe, AZ); Invitrogen, Corp. (Carlsbad, CA); and Illumina, Inc. (San Diego, CA). In certain embodiments, a target biomarker can be measured using a microfluidic card (e.g., TaqMan® Array Microfluidic Card (Applied Biosystems).

### IV. Detection of Target Protein(s)

### A. Detection by Immunoassay

In specific embodiments, the target biomarker(s) of the present invention can be detected/measured by immunoassay. Immunoassay requires biospecific capture reagents/binding agent, such as antibodies, to capture the biomarker. Many antibodies are available commercially. Antibodies also can be produced by methods well known in the art, e.g., by immunizing animals with the biomarkers. Target biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the biomarkers employed herein are quantified by immunoassay, such as enzyme-linked immunoassay (ELISA) technology. In specific embodiments, the levels of expression of the biomarkers are determined by contacting the biological sample with antibodies, or antigen-binding fragments thereof, that selectively bind to the biomarker; and detecting binding of the antibodies, or antigen-binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety.

For example, the level of a target biomarker in a sample can be assayed by contacting the biological sample with an antibody, or antigen-binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target biomarker. A target biomarker can be an entire protein, or a variant or modified form thereof. Kits for the detection of biomarkers as described herein can include precoated strip/plates, biotinylated secondary antibody, standards, controls, buffers, streptavidin-horse radish peroxidase (HRP), tetramethyl benzidine (TMB), stop reagents, and detailed instructions for carrying out the tests including performing standards.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; No. 5,670,637; No. 5,696,249; No. 5,270,163; No. 5,707,796; No. 5,595,877; No. 5,660,985; No. 5,567,588; No. 5,683,867; No. 5,637,459; and No. 6,011,020.

In specific embodiments, the assay performed on the biological sample can comprise contacting the biological sample with one or more capture agents (e.g., antibodies, peptides, aptamer, etc., combinations thereof) to form a biomarker: capture agent complex. The complexes can then be detected and/or quantified.

In one method, a first capture or binding agent such as an antibody that specifically binds the biomarker of interest is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in U.S. Pat. Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different polypeptide biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in U.S. Pat. Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

### B. Detection by Electrochemiluminescent Assay

In several embodiments, the target biomarker(s), and optionally other biomarkers, can be detected by means of an electrochemiluminescent assay developed by Meso Scale Discovery (Gaithersrburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ~620 nm, eliminating problems with color quenching. See U.S. Pat. Nos. 7,497,997; No. 7,491,540; No. 7,288,410; No. 7,036,946; No. 7,052,861; No. 6,977,722; No. 6,919,173; No. 6,673,533; No. 6,413,783; No. 6,362,011; No. 6,319,670; No. 6,207,369; No. 6,140,045; No. 6,090,545; and No. 5,866,434. See also U.S. Pat. Applications Publication Nos. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

### C. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremong, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the biomarkers comprising one or more of HMGA2, PLAG1, KLK7, FNDC4 and CDH3, described herein. In particular embodiments, a few micro-liters of blood serum or plasma are dropped on the chip array. Biomarker proteins present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### V. Treatment of Patients Guided by Target Biomarker Panel

The treatment or therapy can comprise an appropriate treatment modality for a subject having thyroid cancer. Such treatments can comprise one or more of thyroidectomy and radioactive iodine therapy, a TERT inhibitor, a BRAF mutant inhibitor, a MEK inhibitor and combinations of the foregoing. In particular embodiments, therapy comprises a thyroid surgical procedure, which can refer to a surgical procedure involving the thyroid gland. Examples of thyroid surgical procedures include, but are not limited to, a thyroidectomy and a thyroid cancer surgery.

In a specific embodiment, treatment comprises administering a TERT inhibitor. In a more specific embodiment, the TERT inhibitor comprises 2-[(E)-3-naphthen-2-yl but-2-enoylamino]benzoic acid (BIBR1532) and derivatives thereof. See Ward & Autexier, Mol. Pharmacol. 68:779-786, 2005; also J. Biol. Chem. 277(18):15566-72, 2002). TERT modulator antagonists can also include, but are not limited to, TMPvP4 (tetra-(N-methyl-4-pyridyl)porphyrin); telomerase inhibitor IX (MST312); MnTMPyp pentachloride; BPPA; β-Rubromycin; Trichostatin A; Costunolide; Doxorubicin; Suramin Sodum; (-)-Epigallocatchin Gallate (and other catechins); triethylene tetraamine; geldanamycin; 17-(allylamino)-17-demethoxygeldanamycin; and derivatives of the foregoing. In another embodiment, a TERT inhibitor comprises azidothymidine (AZT).

In certain embodiments, treatment comprises administering a BRAF inhibitor. Examples of BRAF inhibitors include Sorafenib (Bay 43-9006, Nexavar), Vemurafenib (PLX4032), BDC-0879, PLX-4720, Dabrafenib (Tafinlar), and Encorafenib (LGX818), RAF265 (CHIR-265) AZ628 and derivatives of the foregoing.

In another embodiment, treatment comprises administering a MEK inhibitor. Examples of MEK inhibitors include trametinib (GSK 1120212), selumetinib (AZD6244), PD184352 (CI1040), PD0325901, RDEA119 (refametinib, BAY 869766), cobimetinib (GDC-0973, RF7420), binimetinib (MEK162, ARRY-162, ARRY-438162), Pimasertib (AS-703026), TAK-733, BI-847325, GDC-0623, PD98059, and derivatives of the foregoing.

In particular embodiments, treatment or therapy can include, but is not limited to, external beam radiation therapy; thyroid hormone therapy including levothyroxine (LEVOXYL, SYNTHROID, and the like); vandetanib (CAPRELSA); cabozantinib (COMETRIQ); sorafenib (NEXAVAR); lenvatinib (LENVIMA); dabrafenib (TAFINLAR); trametinib (MEKINIST); larotrectinib (VITRAKVI); entrectinib (ROZLYTREK); thyroid lobectomy; and lymph node dissection.

Further embodiments include treatment with one or more anti-cancer drugs. The following are lists of anti-cancer drugs that can be used in conjunction with the presently disclosed methods: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil (e.g., 5-fluorouracil); Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### VI. Kits for Detection of Target Biomarkers

All the essential reagents required for detecting and quantifying the target biomarker(s) of the invention may be assembled together in a kit. In some embodiments, the kit comprises a reagent that permits quantification of one or more of HMGA2, PLAG1, KLK7, FNDC4 and CDH3. In some embodiments, the kit comprises: (i) at least one reagent that allows quantification (e.g., determining the abundance, concentration or level) of an expression product of one or more of HMGA2, PLAG1, KLK7, FNDC4 and CDH3 in a biological sample; and optionally (ii) instructions for using the at least one reagent. The kit can further comprise reagents for detection/measurement of other biomarkers.

In the context of the present invention, “kit” is understood to mean a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components contained in the kit. The instructions can be in the form of printed material or in the form of an electronic support capable of storing instructions such that they can be read by a subject, such as electronic storage media, optical media, and the like. Alternatively, or in addition, the media can contain internet addresses that provide the instructions.

The kits may also optionally include appropriate reagents for detection of labels, positive and negative controls, washing solutions, blotting membranes, microtiter plates, dilution buffers and the like. For example, a protein-based detection kit may include an antibody that binds specifically to one or more of HMGA2, PLAG1, KLK7, FNDC4 and CDH3. The kit may also include a target biomarker(s) polypeptide to be used as positive control.

In particular embodiments, the kit is an immunoassay or ELISA kit. The ELISA kit may comprise a solid support, such as a chip, microtiter plate (e.g., a 96-well plate), bead, or resin having biomarker capture reagents attached thereon. The kit may further comprise a means for detecting the biomarker, such as antibodies, and a secondary antibody-signal complex such as horseradish peroxidase (HRP)-conjugated goat anti-rabbit IgG antibody and tetramethyl benzidine (TMB) as a substrate for HRP.

Alternatively, the kit may be provided as an immuno-chromatography strip comprising a membrane on which the antibodies are immobilized, and a detection agent, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be tested by adding a sample such as blood from the patient to the kit and detecting the relevant biomarker(s) conjugated with antibodies, specifically, by a method which comprises the steps of: (i) collecting a biological sample (e.g., blood or FNA) from the patient; (ii) adding the sample from the patient to a diagnostic kit; and, (iii) detecting the biomarker(s) conjugated with antibodies. In this method, the antibodies are brought into contact with the patient sample. If the biomarker(s) are present in the sample, the antibodies will bind to the sample, or a portion thereof. In other kit and diagnostic embodiments, the sample need not be collected from the patient (i.e., it is already collected). Moreover, in other embodiments, the sample may comprise a tissue sample or a clinical sample.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

Alternatively, a nucleic acid-based detection kit may include a primer or probe that specifically hybridizes to a target polynucleotide (HMGA2, PLAG1, KLK7, FNDC4 and CDH3) (e.g., a cDNA of a target or target transcript or the transcript itself). The kit can further include a target biomarker polynucleotide to be used as a positive control. Also included may be enzymes suitable for amplifying nucleic acids including various polymerases (reverse transcriptase, Taq, Sequenase™, DNA ligase etc., depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

In a more specific embodiment, the kit is provided as a PCR kit comprising primers that specifically bind to one or more of the nucleic acid biomarkers described herein. Primers the specifically bind and amplify the target biomarkers described herein include, but are not limited to, HMGA2, PLAG1, KLK7, FNDC4 and CDH3. In specific embodiments, the kit comprises a primer set forth in SEQ ID NOS: 1-2 (HMGA2), SEQ ID NOS:3-4 (PLAG1), SEQ ID NOS:5-6 (KLK7), SEQ ID NOS:7-8 (FNDC4) and/or SEQ ID NOS:9-10 (CDH3). The kit can further comprise a primer for a control such as TPO1 (SEQ ID NOS:11-12). In other embodiments, the kit comprises a primer(s) that binds to a region set forth in SEQ ID NO:14 (HMGA2), SEQ ID NO:16 (PLAG1), SEQ ID NO:18 (KLK7), SEQ ID NO:20 (FNDC4) and SEQ ID NO:22 (CDH3).. The kit can further comprise substrates and other reagents necessary for conducting PCR (e.g., quantitative real-time PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer:biomarker complex. The complexes can then be amplified and detected/quantified/measured to determine the levels of one or more biomarkers. The subject can then be identified as having myocardial injury based on a comparison of the measured levels of one or more biomarkers to one or more reference controls.

The kit can also feature various devices and reagents for performing one of the assays described herein; and/or printed instructions for using the kit to quantify the expression of a target biomarker gene including HMGA2, PLAG1, KLK7, FNDC4 and CDH3.

The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Before the introduction of the NIFTP subtype, the present inventors performed a transcriptome microarray analysis of 125 tumor samples representing the most common epithelial thyroid tumor diagnoses: adenomatoid nodules (AN), follicular adenomas (FA), Hürthle cell adenomas (HA), follicular carcinomas (FC), Hürthle cell carcinomas (HC), FVPTC, and PTC, and identified over 75 transcripts that were differentially expressed between benign and malignant tumors (15). In a follow-up study, we further characterized 14 of these transcripts by a combination of immunohistochemical and quantitative reverse transcription-PCR assays, and identified a candidate 3-gene panel as a potential preoperative diagnostic tool for FNA samples (16).

As described herein, we selected 12 of the 14 genes based on their diagnostic performance in a receiver operating characteristic (ROC) analysis, to determine if newly characterized specific isoforms further improved their diagnostic performance, including cases of the pathologically confirmed NIFTP tumor subtype. We also investigated isoforms of thyroglobulin and thyroid peroxidase (TPO) to identify a thyrocyte-specific load control, since, in contrast to qualitative markers such as mutations, quantitative molecular assessments are complicated by highly variable samples and admixture of non-thyroid derived cells like peripheral blood mononuclear cells (PBMC). Finally, we tested our 5 best performing candidate isoforms directly in intra-operatively obtained FNA samples.

### Materials and Methods

Clinical samples. Under Institutional Review Board approval, thyroid tumor tissue, intraoperative FNA, and blood specimens were collected from patients undergoing thyroid surgery at Johns Hopkins Hospital. Intraoperative FNAs were collected from the tumor with a 25-gauge needle syringe immediately prior to resection and preserved in 95% Ethanol at -20° C., or in RNALater at -80° C. The FNA site was marked intraoperatively to ensure correlation with the final pathological diagnosis. During pathological prosection, an aliquot of tumor tissue was snap frozen in liquid nitrogen and stored at -80° C. Tumor tissue was identified by hematoxylin and eosin staining of frozen tissue sections, and final surgical pathological diagnoses of samples were confirmed by a pathologist. All FVPTCs were re-reviewed to allow reclassification of NIFTPs from FVPTCs where indicated. PBMCs were isolated from patient blood drawn intraoperatively with Ficoll-Paque Plus (GE Healthcare) and stored at -80° C.

RNA isolation and reverse transcription. Total RNA from 80 frozen thyroid tumors (15 ANs, 14 FAs, 10 HAs, 5 NIFTPs, 7 FVPTCs, 7 HCs, 7 FCs, and 15 PTCs) and PBMCs from 31 thyroid patients were isolated with Trizol (Invitrogen) and RNeasy Mini Kit (Qiagen) following the manufacturer’s instructions. cDNA was synthesized by reverse transcription with 500 ng of total RNA using SuperScript III reverse transcriptase (Invitrogen). Total RNA from single pass FNA samples was isolated using GenElute Single Cell RNA Purification Kit (Sigma-Aldrich) following the manufacturer’s instructions. Because of the variable number of thyroid cells in individual FNA samples, the entire total RNA elution volume (11 µl) was used for cDNA synthesis.

PCR of thyroid tumor tissue and PBMC samples. Each PCR assay was performed with 5% of total cDNA using Platinum Taq DNA polymerase (Invitrogen) following the manufacturer’s instructions. Amplified DNA was analyzed by agarose gel electrophoresis (see below) for 22 isoforms from 12 genes, CEACAM6, CDH3, DIRAS3, DPP4, FNDC4, HMGA2, KLK7, MRC2, SFN, c-KIT, PRSS3, and PLAG1. Additionally, 4 isoforms of thyroglobulin and thyroid peroxidase (TPO) were assessed as potential thyrocyte-specific load control on solid thyroid tumor and PBMC samples. Glyceraldehyde-3-phosphate dehydrogenase (GapDH, NM_002046) served as a total RNA load control.

Real-time quantitative PCR (qPCR) on FNA samples. Selected target and reference gene isoforms were tested by real-time qPCR on 159 FNA samples from 6 confirmed surgical histological subtypes of thyroid tumors; AN, FA, HA, NIFTP, FVPTC, and PTC. (See Table 5 for PCR primer sequences.) Gene expression was quantitated in duplicate using 5% of total cDNA in each assay and Power SYBR Green Master Mix (Applied Biosystems) on a Bio-Rad iQ5 thermal cycler for 40 cycles. The Ct values of the duplicates were averaged. Serial dilutions of frozen tumor RNA were used to establish the threshold of detectability of the selected thyrocyte-specific reference gene in the assay, and an amplification threshold cycle (Ct) value > 30 was chosen to exclude sample from further analysis for lack of sufficient thyrocytes.

Data analysis. For the initial selection of candidate gene isoforms on frozen tumor tissue samples, semi-quantitative densitometry using BioRad Quantity One image analysis software was used to select the isoforms showing the highest levels of differential expression between cancer and non-cancer samples on agarose gel electrophoresis images. Expression levels of each isoform were assessed by normalizing to the RNA load control gene GapDH and then z-transformed, so all genes could be assessed on the same scale.

For the subsequent qPCR analysis of FNA samples, the relative expression of each target gene was calculated with respect to the reference load control gene TPO1 [ΔCt = Ct (target) - Ct (reference)] and then z-transformed for assessment. When a target gene was undetectable after 40 cycles, the sample was assigned the maximum observed ΔCt value + 5% of the standard deviation of the ΔCt values observed for that gene across all samples.

The ability of the candidate genes to distinguish between malignant and benign thyroid tumor subtypes was evaluated using ROC analysis. Overall performance was measured as the AUC.

We then applied Bayes Rule to estimate the positive predictive value (PPV) and negative predictive value (NPV) from our observed sensitivity and specificity using the tumor prevalence rates reported by Steward et al (17).

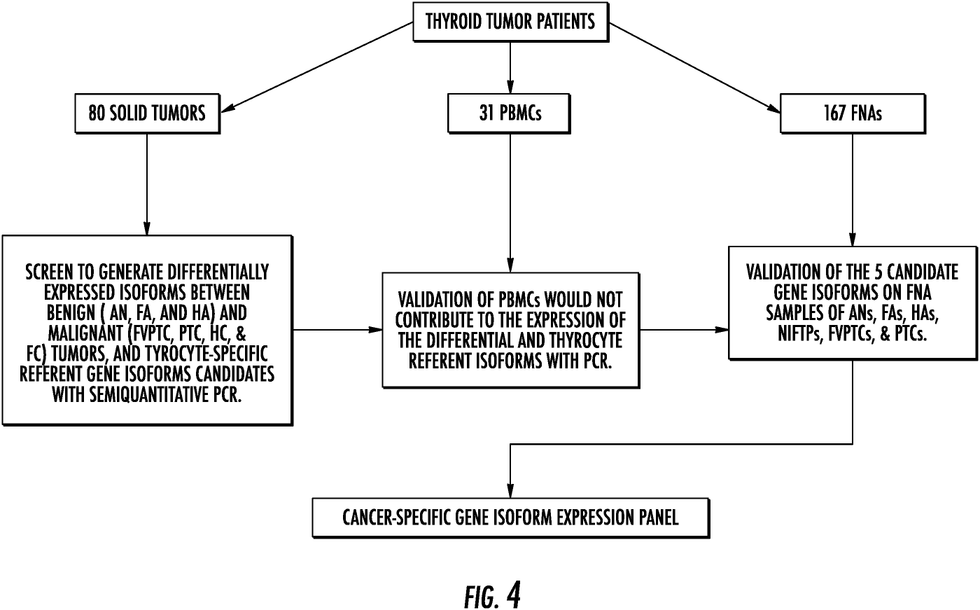

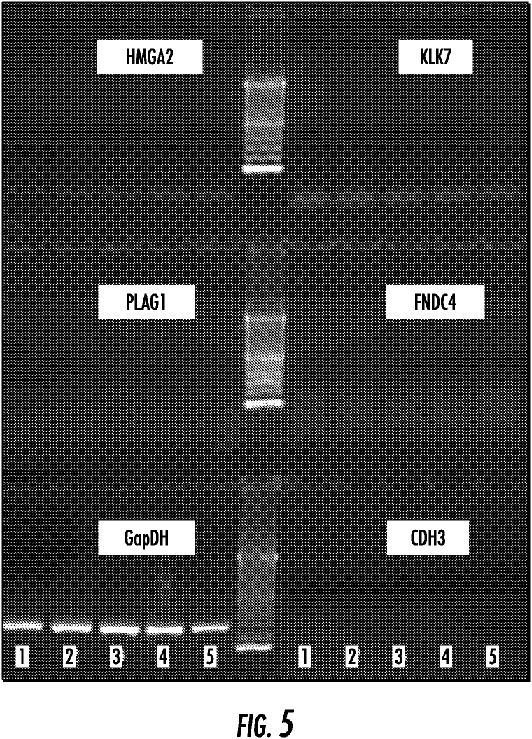

The overall workflow of the study in shown in FIG. 4.

### Results

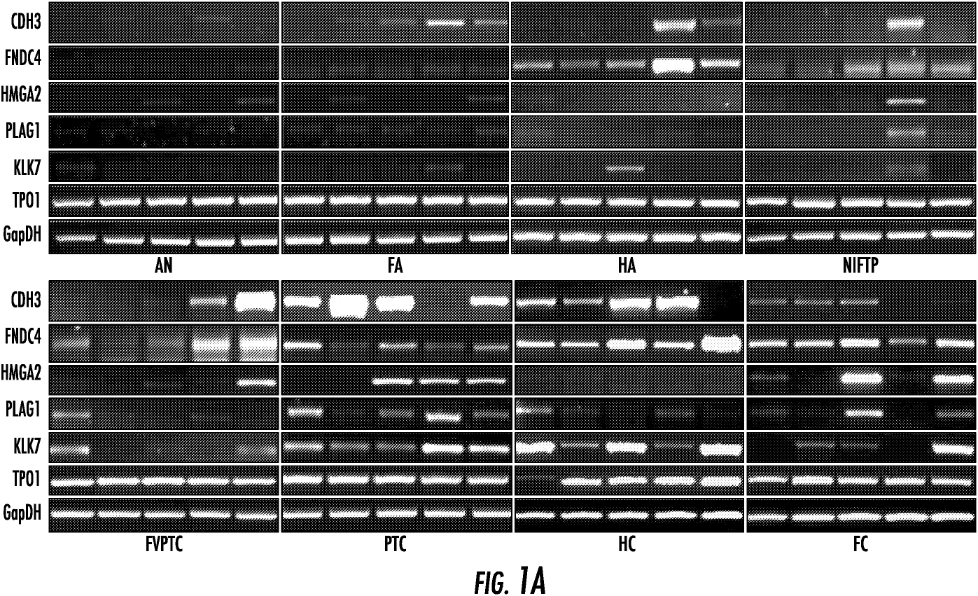

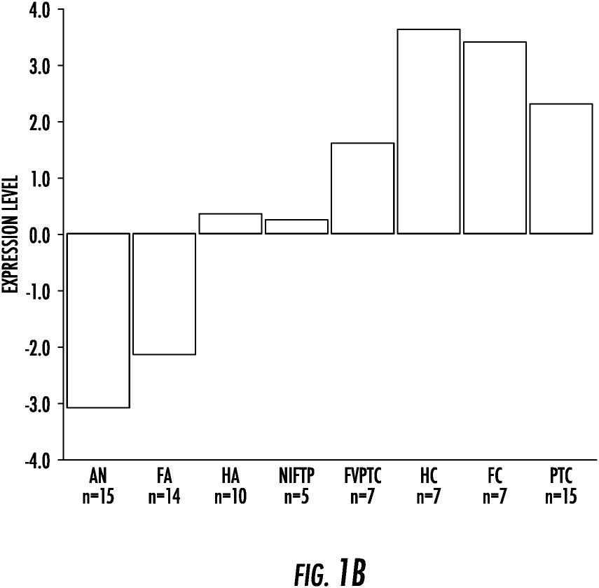

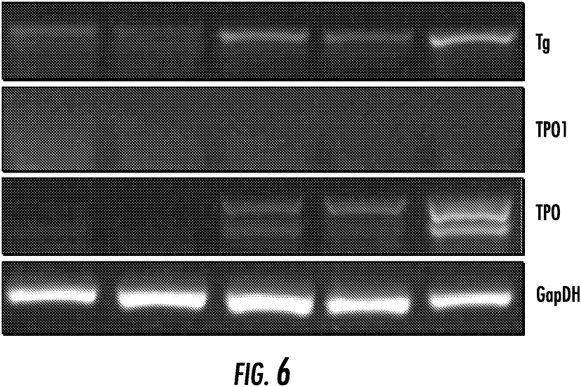

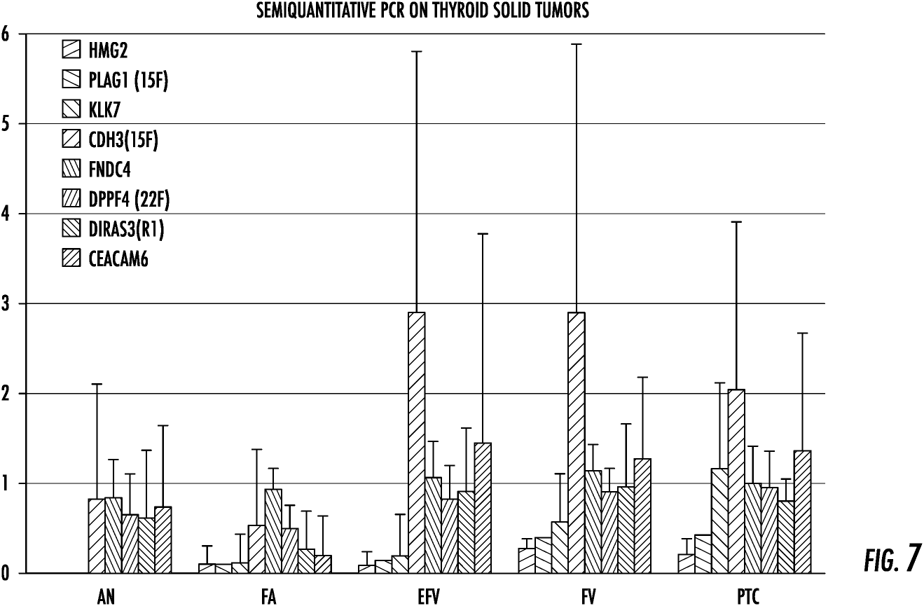

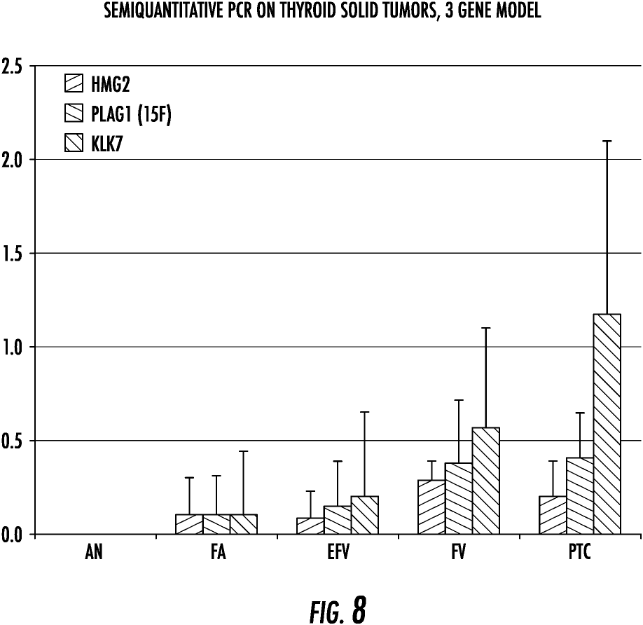

Candidate gene isoform selection by reverse transcription-PCR using tumor tissues and PBMCs. The 22 selected transcript isoforms were characterized on frozen tumor tissue samples from 15 ANs, 14 FAs, 10 HAs, 5 NIFTPs, 7 FVPTCs, 7 HCs, 7 FCs, and 15 PTCs, using semi-quantitative PCR. Five transcript isoforms from the following genes: CDH3, FNDC4, HMGA2, KLK7, PLAG1 showed the most differential expression among thyroid cancers, NIFTPs, and benign tumors (Table 1, FIG. 1), as determined by image analysis of agarose gel electrophoresis of the PCR products. Importantly, none of the isoforms were detectable in PBMCs from 31 patients (FIG. 5). Among the candidate thyrocyte load-control isoforms of TPO and thyroglobulin tested, TPO1 was the only isoform not detectable in PBMCs (FIG. 6) and stably expressed across the well-differentiated thyroid tumor subtypes (FIG. 1A). Therefore, TPO1 was selected as the load control for thyroid cell content of FNA samples.

One recurrent metastatic HC showed markedly reduced TPO1 levels (FIG. 1A). No FNA samples of this case were available for testing, however.

Validation of the 5 candidate gene isoforms on an independent cohort of FNA samples. Seven (4.4%) of the 159 FNA samples tested had no detectable TPO1 expression with 40 qPCR cycles and, were therefore excluded from the study. Serial dilutions of thyroid tissue-derived RNA revealed that TPO1 was detectable at 30 qPCR cycles using a minimum of 370 pg of total RNA, which corresponds to the total RNA of approximately 12-36 cells (18), lower than the cytopathological threshold of minimal thyrocyte content in FNA samples (6 clusters of at least 10 follicular epithelial cells on 2 or more slides)(19). We therefore selected a TPO1 threshold Ct value of 30 to include FNA samples for our study, a criterion met by a total of 137 of 159 (86.2%) FNA samples tested. There was no significant difference in the fail rates across the diagnostic subgroups tested. Table 2 summarizes the patient information for the FNAs used.

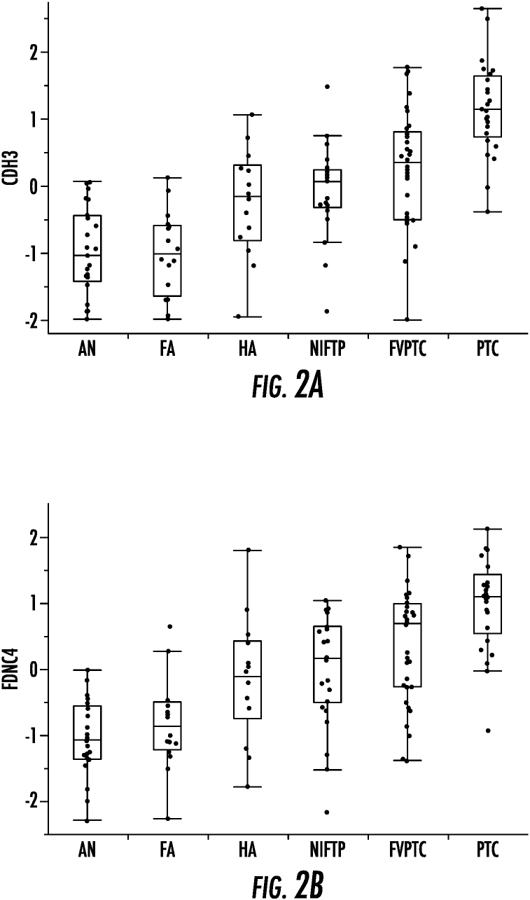

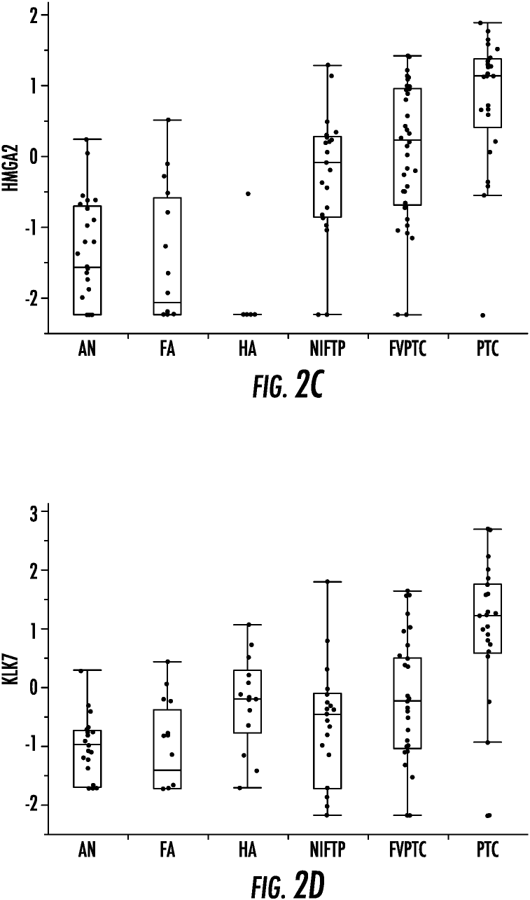

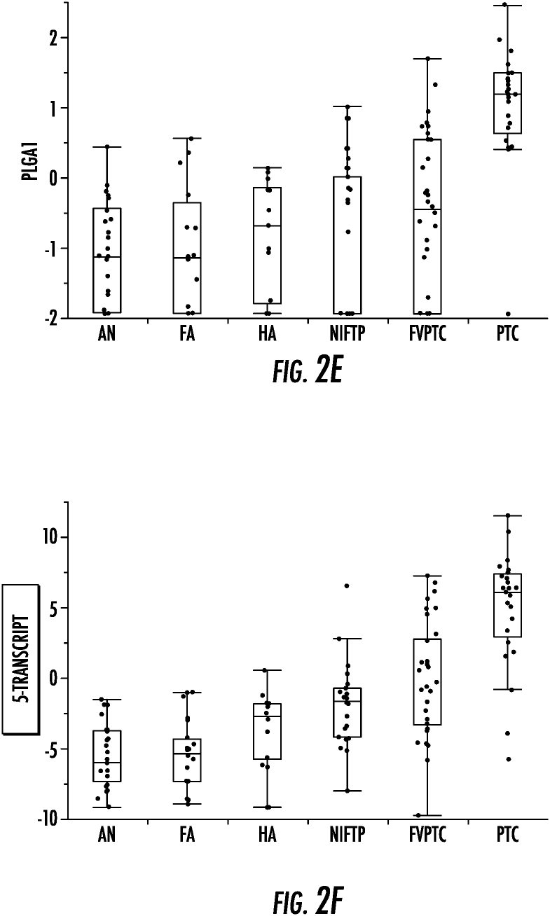

The TPO1-normalized ΔCt data were z-transformed to create a z-ΔCt score for the expression of each target gene. FIG. 2 shows the expression profiles of the selected CDH3, FNDC4, HMGA2, KLK7, PLAG1 isoforms and the composite z-ΔCt score in the 6 thyroid tumor subtypes tested (AN, FA, HA, NIFTP, FVPTC, and PTC). Each individual isoform showed higher expression in malignant thyroid tumors, than in benign. When summing the 5 transcripts, the composite z-ΔCt score exhibited a differential profile among thyroid tumors (FIG. 2F).

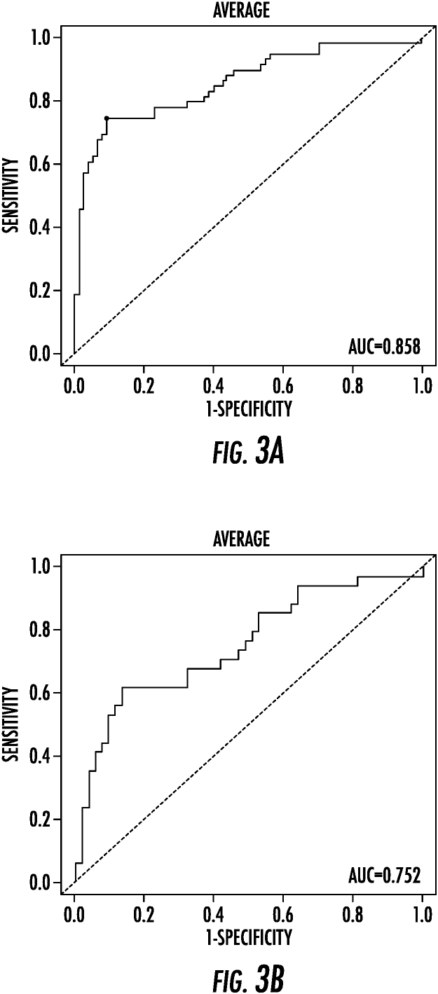

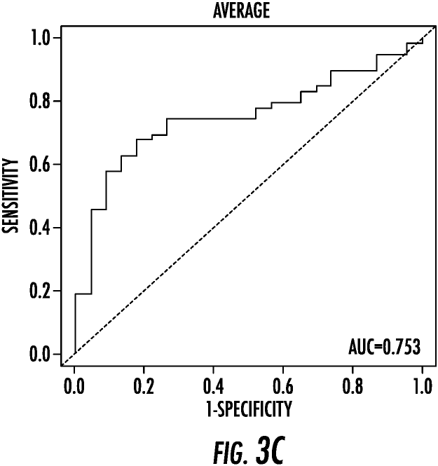

An ROC analysis (FIG. 3A) was used to evaluate the ability of our 5-gene isoform expression panel to differentiate benign tumors (AN, FA, HA, and NIFTP, n = 78) from malignant tumors (FVPTC and PTC, n = 59). Overall performance was measured as the AUC, which was 0.86. Several combinations of sensitivity and specificity, representing points along the ROC curve, are shown in Table 3 as well. One of these combinations is highlighted on the ROC curve and on the strip-plots in FIG. 2. Corresponding to a threshold for the composite expression score (z-ΔCt) of -1, this value was chosen to maximize sensitivity (75%) while controlling specificity above 90% (actual value = 91%). Forty-four of 59 (74.6%) malignant thyroid tumors had a composite score > -1, (23/25, 92.0% of PTCs; 21/34, 61.8% of FVPTCs), while only 7 out of 78 (9.0%) benign and NIFTP nodules had a score > -1 (6/23, 26.1% of NIFTPs; 1/14, 7.1% of HAs; 0/16 of FAs; and 0/25, 0% of ANs; p < 0.0001).

We used the prevalence rates from a previous large multicenter study (17) to apply Bayes Rule to estimate the NPV and PPV from our observed sensitivity and specificity, resulting in an NPV of 91% and a PPV of 74% (Table 3). Variations of this calculation, assuming malignant sample prevalence rates ranging from 20% to 30%, are shown in Table 6.

The panel also significantly differentiated the NIFTPs from the malignant PTCs and FVPTCs (26.1% of NIFTPs versus 74.6% of cancers with scores > -1, p = 0.0002). Further, the comparison between NIFTP versus invasive FVPTC showed a statistically significant separation (p < 0.05).

### Discussion

In the past decade, molecular testing has emerged as a promising method to increase the accuracy of the preoperative diagnosis of malignant thyroid tumors. Several molecular diagnostic tests, including RNA based gene expression and multi-panel mutation genotyping analysis are commercially available for clinical use (17,20). Available tests remain, however, limited by relatively low specificity and PPV (21,22). Furthermore, the recent reclassification of a subgroup of malignant FVPTC to clinically “benign” NIFTP has further complicated the situation, since prior publications assessing the performance of commercially available molecular tests were based upon NIFTP being categorized as malignant. Indeed, most NIFTPs were reported as suspicious/malignant by expression based Afirma or mutation and gene fusion based ThyroSeq in several studies (14,21,23,24).

Our group previously carried out a series of studies to identify genetic markers for distinguishing cancer from benign thyroid tumors using genome-wide gene expression arrays (15,16). In this study, we have developed a molecular test for evaluating preoperative thyroid FNAs taking NIFTP lesions into consideration, by further characterizing isoforms of our previously profiled gene candidates.

Taking advantage of the improved annotation of the human genome over the last decade, we first explored expression of a broad range of splice variants of our 12 gene set in frozen thyroid tumor samples to select candidates which were differentially expressed in benign and malignant neoplasms. In this study, the 5 isoforms of CDH3, FNDC4, HMGA2, KLK7, and PLAG1 we identified showed potential in differentiating different thyroid tumor subtypes, and importantly, were chosen because they were not expressed in PBMCs. In FNA samples, quantitative molecular tests must address the contribution from PBMCs. Positive or negative selection using antibody-coated magnetic beads can minimize their contribution, but their use decreases overall assay sensitivity (data not shown). In the absence of selection, the load control reference gene needs to reflect the number of thyrocytes in FNA samples rather than a standard total RNA content measure. In our study, we tested two isoforms of TPO and two of thyroglobulin. Only TPO1 was found to have constant levels of expression across differentiated thyroid tumor subtypes (FIG. 1A) and importantly, was undetectable by PCR after 40 cycles in any of the 31 PBMC samples obtained perioperatively (FIG. 6). We did observe, however, a decrease in TPO 1 expression in one large HC metastasis, possibly a consequence of loss of differentiation in this recurrent advanced tumor.

The 5-isoform panel was tested using our intraoperative FNA samples. In this study, only a single pass of needle aspirate from each tumor was used for our analysis. To ensure the reliability of the assay, only samples reaching a TPO1 threshold of detectability of 30 cycles or less were included in this study. Only 7 (4.4%) of FNA samples showed no detectable TPO1, and 137 (86.2%) of 159 FNA samples met our threshold and produced reliable gene expression profiles. This compares favorably with reported 2-20% of cases yielding cytopathologically non-diagnostic results with two to five FNA passes (25).

In our study, quantitative PCR data generated from 137 qualified FNA samples demonstrated a differential expression among benign and malignant thyroid tumors. The ROC analysis shows our 5-isoform panel has an 86% ability to distinguish benign thyroid tumors (AN, FA, and HA) and NIFTP from malignant tumors (FVPTC and PTC), with a specificity of 91% at a sensitivity of 75%, resulting in an NPV of 91% and a PPV of 74%.

The thyroid follicular-patterned lesions, FA, NIFTP, and FVPTC, contribute the most to the indeterminate cytologies. Molecularly, they all are frequently associated with RAS mutations (26). Currently, histologic evaluation of capsule and vascular invasion is necessary for diagnosis of NIFTP. Thus, an accurate diagnosis of NIFTP is impossible by preoperative cytology or mutation based molecular tests. Nevertheless, NIFTP is an indolent lesion with <1% risk of recurrence (27), and should not be treated as thyroid cancer, although it may warrant resection as potential premalignant lesion.

The NIFTP classification is new. Currently, the reclassified indolent NIFTP is still considered as a surgical disease by most endocrinologists. Its separation from malignant FVPTC may significantly impact clinical treatment decisions, leading to lesser surgical and other ablative procedures, and potentially simple observation as therapeutic options, which are currently under active investigation. More studies, especially prospective long-term follow-up studies, are needed to evaluate its behavior, progression, and optimal management. Finding tools for accurate preoperative identification of NIFTP will promote the study and management for this lesion. The data presented here show our newly developed 5-isoform panel may reduce cytologically and molecularly indeterminate diagnoses.

Our study also has a number of limitations, foremost the number of available intraoperative FNAs, which also do not exactly replicate the standard preoperative diagnostic FNAs typically obtained percutaneously in a clinic setting. We limited ourselves to epithelial thyroid tumors, and were unable to obtain FNA samples from the infrequent FC and HC cases encountered in the timespan samples were collected for this study. Therefore, although the available FC and HC tissue samples had high scores, suggesting the selected isoform panel may do well diagnostically, the performance of the panel in these cases remains unknown for FNAs.

### Conclusions

In conclusion, we have developed a 5-transcript model combining specific splice variants of HMGA2, PLAG1, KLK7, FNDC4, and CDH3 to better characterize thyroid nodules using the technically challenging but clinically relevant diagnostic FNA samples. Further validation trials will be needed to develop this panel as a diagnostic assay to guide preoperative surgical decision making for thyroid tumors.

TABLE 1

Gene transcript variants selected

| | | | |
|---|---|---|---|
| Symbol | Reference | Gene name | Isoform |
| CDH3 | NM_001793.5 | Cadherin 3 | transcript variant 1 |
| FNDC4 | NM_022823.2 | Fibronectin type III domain containing 4 | transcript variant 1 |
| HMGA2 | NM_003483.4 | High mobility group AT-hook 2 | transcript variant 1 |
| KLK7 | NM_005046.3 | Kallikrein related peptidase 7 | transcript variant 1 |
| PLAG1 | NM_002655.2 | PLAG1 zinc finger | transcript variant 1 |
| TPO 1 | NM_000547.5 | Thyroid peroxidase | transcript variant 1 |

TABLE 2

Patient information of the FNA study cohort

| | AN | FA | HA | NIFTP | FVPTC | PTC |
|---|---|---|---|---|---|---|
| Sample size, n | 25 | 16 | 14 | 23 | 34 | 25 |
| Sex, M/F | 8/17 | 4/12 | 3/11 | 7/16 | 5/29 | 9/16 |
| Age, years | 48.1 (27-68) | 46.1 (18-74) | 53.6 (29-80) | 50.1 (27-72) | 44.4 (19-76) | 41.5 (18-59) |
| Nodule size, cm | 3.2 (0.8-7.8) | 2.8 (1.0-7.5) | 3.1 (1.1-7.0) | 2.9 (0.9-8.0) | 2.5 (0.6-6.0) | 2.4 (0.6-5.0) |
| Bethesda I, n | 1 | | | | | |
| Bethesda II, n | 11 | 3 | | 3 | 4 | |
| Bethesda III, n | 5 | 1 | | 12 | 7 | 1 |
| Bethesda IV, n | 7 | 10 | 13 | 2 | 8 | 1 |
| Bethesda V, n | 1 | | | 4 | 9 | 5 |
| Bethesda VI, n | | 1 | | 2 | 6 | 18 |
| Indeterminate cytology*, n | | 1 | 1 | | | |

AN, adenomatoid nodule; FA, follicular adenomas; HA, Hürthle cell adenoma; NIFTP, noninvasive follicular thyroid neoplasm with papillary-like nuclear features; FVPTC, follicular variant of papillary thyroid carcinoma; PTC, papillary thyroid carcinoma.
^{∗}Clinical FNA obtained before current Bethesda. classification available.

TABLE 3

Performance of the 5-transcript panel in benign vs. malignant FNAs

| Sensitivity, % | Specificity, % | PPV, % | NPV, % |
|---|---|---|---|
| 78 | 76 | 53 | 91 |
| 75 | 81 | 58 | 90 |
| 75 | 86 | 65 | 91 |
| 75 | 91 | 74 | 91 |
| 61 | 96 | 85 | 88 |
| 19 | 100 | 100 | 78 |

TABLE 4

Performance of the 5-transcript panel in differentiating benign vs. malignant follicular lesions or NIFTP vs. malignant FNAs

| NIFTP, HA, and FA vs. FVPTC | | NIFTP vs. FVPTC and PTC | |
|---|---|---|---|
| Sensitivity, % | Specificity, % | Sensitivity, % | Specificity, % |
| 59 | 75 | 69 | 78 |
| 56 | 81 | 68 | 83 |
| 53 | 87 | 63 | 87 |
| 44 | 91 | 58 | 91 |
| 29 | 96 | 46 | 96 |
| 9 | 100 | 19 | 100 |

TABLE 5

Primer sequences of the 5 transcripts and reference gene

| | | |
|---|---|---|
| Gene | Forward primer | Reverse primer |
| HMGA2 | AAGCCACTGGAGAAAAACGG (SEQ ID NO:1) | CTCTTCGGCAGACTCTTGTGA (SEQ ID NO:2) |
| PLAG 1 | TGCTTCATTCTGTGACGGTCTATT (SEQ ID NO:3) | ACTTTGATCTTAGCCAGTCCCATT (SEQ ID NO:4) |
| hKLK7 | CCCTGCTCAGTGGCAATCA (SEQ ID NO:5) | CTGTCGCCCAGCGTATCA (SEQ ID NO:6) |
| hFNDC4 | TCACTCACCTCAGAGCCAAC (SEQ ID NO:7) | TTCACCTCCCGAATCACAC (SEQ ID NO:8) |
| CDH3 | CATCAGCGTCATCTCCAGTG (SEQ ID NO:9) | ATCAGTGACCGTCAGCCTCT (SEQ ID NO:10) |
| TPO1 | ACAACAGAGACCACCCCAGATG (SEQ ID NO:11) | GCCATCAGGAGGTCAGAATAGC (SEQ ID NO:12) |

TABLE 6

PPV and NPV of the 5-transcript panel at different cancer prevalence rates

| 20% malignant | | 30% malignant | |
|---|---|---|---|
| NPV, % | PPV, % | NPV, % | PPV, % |
| 93 | 44 | 90 | 58 |
| 93 | 49 | 89 | 62 |
| 93 | 57 | 89 | 69 |
| 93 | 68 | 89 | 78 |
| 91 | 80 | 85 | 87 |
| 83 | 100 | 74 | 100 |

### REFERENCES

1. Surveillance Epidemiology and End Results program (SEER). Cancer Stat Facts: Thyroid Cancer. National cancer institute. 2018.
2. Ali SZ, Cibas E. The Bethesda System for Reporting Thyroid Cytopathology. 2nd ed. Ali SZ, Cibas ES, editors. Springer International Publishing; 2018. XV, 236.
3. Haugen BR, Alexander EK, Bible KC, Doherty GM, Mandel SJ, Nikiforov YE, et al. 2015 American Thyroid Association Management Guidelines for Adult Patients with Thyroid Nodules and Differentiated Thyroid Cancer: The American Thyroid Association Guidelines Task Force on Thyroid Nodules and Differentiated Thyroid Cancer. Thyroid. 2016 Jan 1;26(1):1-133.
4. Bongiovanni M, Giovanella L, Romanelli F, Trimboli P. Cytological Diagnoses Associated with Noninvasive Follicular Thyroid Neoplasms with Papillary-Like Nuclear Features According to the Bethesda System for Reporting Thyroid Cytopathology: A Systematic Review and Meta-Analysis. Thyroid. 2019;29(2):222-8.
5. Nikiforov YE, Baloch ZW. Clinical validation of the ThyroSeq v3 genomic classifier in thyroid nodules with indeterminate FNA cytology. Cancer Cytopathol [Internet]. 2019 Apr 27 [cited 2021 Apr 15];127(4):225-30. Available from: https://onlinelibrary.wiley.com/doi/abs/10.1002/cncy.22112
6. Endo M, Nabhan F, Porter K, Roll K, Shirley LA, Azaryan I, et al. Afirma Gene Sequencing Classifier Compared with Gene Expression Classifier in Indeterminate Thyroid Nodules.
7. Lupo MA, Walts AE, Sistrunk JW, Giordano TJ, Sadow PM, et al. Multiplatform molecular test performance in indeterminate thyroid nodules. 2020;
8. Labourier E, Fahey TJ. Preoperative molecular testing in thyroid nodules with Bethesda VI cytology: Clinical experience and review of the literature. Diagn Cytopathol. 2021 Apr 1;49(4):E175-80.
9. Livhits MJ, Zhu CY, Kuo EJ, Nguyen DT, Kim J, Tseng C-H, et al. Effectiveness of Molecular Testing Techniques for Diagnosis of Indeterminate Thyroid Nodules A Randomized Clinical Trial Visual Abstract Supplemental content. JAMA Oncol [Internet]. 268AD;7(1):70-7. Available from: https://jamanetwork.com/
10. Al-Qurayshi Z, Deniwar A, Thethi T, Mallik T, Srivastav S, Murad F, et al. Association of malignancy prevalence with test properties and performance of the gene expression classifier in indeterminate thyroid nodules. JAMA Otolaryngol - Head Neck Surg [Internet]. 2017;143(4):403-8. Available from: https://www.mendeley.com/catalogue/association-malignancy-prevalence-test-properties-performance-gene-expression-classifier-indetermina/
11. Shrestha RT, Evasovich MR, Amin K, Radulescu A, Sanghvi TS, Nelson AC, et al. Correlation between Histological Diagnosis and Mutational Panel Testing of Thyroid Nodules: A Two-Year Institutional Experience. Thyroid. 2016;26(8):1068-76.
12. Parajuli S, Jug R, Ahmadi S, Jiang XS. Hürthle cell predominance impacts results of Afirma gene expression classifier and ThyroSeq molecular panel performance in indeterminate thyroid nodules. Diagn Cytopathol. 2019;47(11):1177-83.
13. Bose S, Sacks W, Walts AE. Update on Molecular Testing for Cytologically Indeterminate Thyroid Nodules. Adv Anat Pathol. 2019;26(1):114-123.
14. Jug RC, Datto MB, Jiang XS. Molecular Testing for Indeterminate Thyroid Nodules: Performance of the Afirma Gene Expression Classifier and ThyroSeq Panel. Cancer Cytopathol. 2018;126(7):471-80.
15. Prasad NB, Somervell H, Tufano RP, Dackiw APB, Marohn MR, Califano JA, et al. Identification of genes differentially expressed in benign versus malignant thyroid tumors. Clin Cancer Res. 2008;14(11):3327-37.
16. Prasad NB, Kowalski J, Tsai HL, Talbot K, Somervell H, Kouniavsky G, et al. Three-gene molecular diagnostic model for thyroid cancer. Thyroid. 2012;22(3):275-84.
17. Steward DL, Carty SE, Sippel RS, Yang SP, Sosa JA, Sipos JA, et al. Performance of a Multigene Genomic Classifier in Thyroid Nodules with Indeterminate Cytology: A Prospective Blinded Multicenter Study. JAMA Oncol. 2019 Feb 1;5(2):204-12.
18. Nygaard V, Hovig E. Options available for profiling small samples: A review of sample amplification technology when combined with microarray profiling. Nucleic Acids Res [Internet]. 2006;34(3):996-1014. Available from: https://www.ncbi.nlm.nih.gov/pubmed/16473852
19. Cibas ES, Ali SZ. The 2017 Bethesda System for Reporting Thyroid Cytopathology. Thyroid. 2017;27(11):1341-6.
20. Nikiforov YE. Role of molecular markers in thyroid nodule management: Then and now. Endocr Pract. 2017;23(8):979-88.
21. Samulski TD, LiVolsi VA, Wong LQ, Baloch Z. Usage trends and performance characteristics of a “gene expression classifier” in the management of thyroid nodules: An institutional experience. Diagn Cytopathol. 2016;44(11):867-73.
22. Brauner E, Holmes BJ, Krane JF, Nishino M, Zurakowski D, Hennessey J V., et al. Performance of the Afirma Gene Expression Classifier in Hürthle Cell Thyroid Nodules Differs from Other Indeterminate Thyroid Nodules. Thyroid [Internet]. 2015;25(7):789-96. Available from: https://www.ncbi.nlm.nih.gov/pubmed/25962906
23. Sahli ZT, Umbricht CB, Schneider EB, Zeiger MA. Thyroid Nodule Diagnostic Markers in the Face of the New NIFTP Category: Time for a Reset? Thyroid. 2017;27(11):1393-9.
24. Jiang XS, Harrison GP, Datto MB. Young Investigator Challenge: Molecular testing in noninvasive follicular thyroid neoplasm with papillary-like nuclear features. Cancer Cytopathol. 2016;124(12):893-900.
25. Cibas ES, Ali SZ. The Bethesda system for reporting thyroid cytopathology. Am J Clin Pathol. 2009;19(11):1159-65.
26. Esapa CT, Johnson SJ, Kendall-Taylor P, Lennard TWJ, Harris PE. Prevalence of Ras mutations in thyroid neoplasia. Clin Endocrinol (Oxf). 1999;50(4):529-35.
27. Hodak S, Tuttle RM, Maytal G, Nikiforov YE, Randolph G. Changing the Cancer Diagnosis: The Case of Follicular Variant of Papillary Thyroid Cancer - Primum Non Nocere and NIFTP. Thyroid. 2016 Jul 1;26(7):869-71.

### Example 2: Assays for Sample Preparation and Detection of 5 Biomarkers and 1 Load-Control Marker.

Samples. Fine needle aspiration (FNA) biopsy samples from the archival FNA collection, as well as from freshly collected samples. In certain embodiments, due to the heterogeneic property of the tumors, 3 FNAs from each tumor are obtained.

Total RNA isolation. Total RNA was extracted from each tumor FNAs using GenElute Single Cell RNA Purification kit (Sigma-Aldrich) and following the manufacturer’s instruction. Use 15 µl of elution buffer for the final elution. The final RNA volume is about 14 µl.

Reverse transcription. Reverse transcription was performed with all the 14 µl of total RNA from each tumor and Oligo(dT) Primers using SuperScript III reverse transcriptase (Invitrogen).

Real-time Quantitative Polymerase Chain Reaction (qPCR). The presence of thyroid epithelial cells was checked by measuring the expression of Homo sapiens thyroid peroxidase transcript variant 1 (TPO1) using SYBR qPCR reagent. The TPO 1 positive samples were further analyzed using qPCR for the expression levels of Homo sapiens high mobility group AT-hook 2 (HMGA2) transcript variant 1, Homo sapiens PLAG1 zinc finger (PLAG1) transcript variant 1, Homo sapiens kallikrein related peptidase 7 (KLK7) transcript variant 1, Homo sapiens fibronectin type III domain containing 4 (FNDC4), and Homo sapiens cadherin 3 type 1 P-cadherin (placental) (CDH3). The present inventors have tested and confirmed that these five genes are undetectable in white blood cells, which are unavoidable in FNA-derived materials. The expression level of each of the five genes was determined using TPO 1 as a thyroid epithelial cell reference gene (load control) (Table 5, above).

### Regions for the Primer Design:

Homo sapiens high mobility group AT-hook 2 (HMGA2) transcript variant 1, NM_003483.5 (SEQ ID NO:13):

aagacccaaa ggcagcaaaa acaagagtcc ctctaaagca gctcaaaaga aagcagaagc cactggagaa aaacggccaa
gaggcagacc taggaaatgg ccacaacaag ttgttcagaa gaagcctgct caggaggaaa ctgaagagac atcctcacaa
gagtctgccg aagaggacta gggggcgcca acgttcgatt tctacctcag cagcagttgg atcttttgaa gggagaagac
actgcagtga ccacttattc tgtattgcca tggtctttcc actttcatct ggggtggggt gggggagggg ggggtggggt
ggggagaaat cacataacct taaaaaggac (SEQ ID NO: 14).

Homo sapiens PLAG1 zinc finger (PLAG1) transcript variant 1, NM_002655.3 (SEQ ID NO:15):

gttgcctctt ggtgctgcct tggccgtatt tggcacccag aatgcttcat tctgtgacgg tctattaata aggttgcctt gctagagttt
ggagcagggc ctcagattgg ccaaaatggg aaggattgga ttccactctc ttccacgaag agtcaatggg actggctaag
atcaaagtct gaggcttttt ccatcagtaa tcagtccctt tttgctttct tttacgacca catgaaactt gagaagccac ctaaagctat
atcatttagt ggagttgggc agttcccaag tgtccaacaa gaaggcctgg tttaggctgc gatggccact gtcattcctg gtgatttgtc
agaagtaaga (SEQ ID NO:16).

Homo sapiens kallikrein related peptidase 7 (KLK7) transcript variant 1, NM_005046.4 (SEQ ID NO: 17):

ggtgacaaga ttattgatgg cgccccatgt gcaagaggct cccacccatg gcaggtggcc ctgctcagtg gcaatcagct
ccactgcgga ggcgtcctgg tcaatgagcg ctgggtgctc actgccgccc actgcaagat gaatgagtac accgtgcacc
tgggcagtga tacgctgggc gacaggagag ctcagaggat caaggcctcg aagtcattcc gccaccccgg ctactccaca
cagacccatg ttaatgacct catgctcgtg aagctcaata gccaggccag gctgtcatcc atggtgaaga aagtcaggct
gccctcccgc tgcgaacccc ctggaaccac ctgtactgtc (SEQ ID NO:18).

Homo sapiens fibronectin type III domain containing 4 (FNDC4), NM_022823.3 (SEQ ID NO:19):

accggcctcc ctctcctgtg aatgtgacgg tcactcacct cagagccaac tcggccactg tgtcctggga cgtcccagaa
ggcaacatcg tcattggcta ctccatttcc cagcaacggc agaatggccc cgggcagcgt gtgattcggg aggtgaacac
caccacccgg gcctgtgccc tctggggcct ggctgaagac agtgactaca cagtgcaggt caggagcatc ggccttcggg
gagagagtcc cccagggccc cgggtgcact tccgaactct caagggttct gaccggctac cttcaaacag ttcaagccca
(SEQ ID NO:20).

Homo sapiens cadherin 3 type 1 P-cadherin (placental) (CDH3), NM_001793.6 (SEQ ID NO:21):

tctgtgatgc aggtgacagc cacggatgag gatgatgcca tctacaccta caatggggtg gttgcttact ccatccatag
ccaagaacca aaggacccac acgacctcat gttcaccatt caccggagca caggcaccat cagcgtcatc tccagtggcc
tggaccggga aaaagtccct gagtacacac tgaccatcca ggccacagac atggatgggg acggctccac caccacggca
gtggcagtag tggagatcct tgatgccaat gacaatgctc ccatgtttga cccccagaag tacgaggccc atgtgcctga
gaatgcagtg ggccatgagg tgcagaggct gacggtcact gatctggacg cccccaactc accagcgtgg cgtgccacct
accttatcat gggcggtgac gacggggacc attttaccat caccacccac cctgagagca accagggcat cctgacaacc
(SEQ ID NO:22).

Homo sapiens thyroid peroxidase transcript variant 1 (TPO1), NM_000547.5 (SEQ ID NO:23):

atgctttatc agaagatctg ctgagcatca ttgcaaacat gtctggatgt ctcccttaca tgctgccccc aaaatgccca aacacttgcc
tggcgaacaa atacaggccc atcacaggag cttgcaacaa cagagaccac cccagatggg gcgcctccaa cacggccctg
gcacgatggc tccctccagt ctatgaggac ggcttcagtc agccccgagg ctggaacccc ggcttcttgt acaacgggtt
cccactgccc ccggtccggg aggtgacaag acatgtcatt caagtttcaa atgaggttgt cacagatgat gaccgctatt
ctgacctcct gatggcatgg ggacaataca tcgaccacga catcgcgttc acaccacaga gcaccagcaa agctgccttc
gggggagggg ctgactgcca gatgacttgt gagaaccaaa acccatgttt tcccatacaa (SEQ ID NO:24).

### Example 3: Development of a High Sensitivity and Specificity Test to Evaluate Preoperative Thyroid Fine Needle Aspiration Cytology Material.

The goal of this Example was to develop a high sensitivity and specificity test to evaluate preoperative thyroid fine needle aspiration (FNA) cytology material. FNAs are obtained as an outpatient procedure and are considered standard of practice in the initial evaluation of thyroid nodules. Although FNA cytology is the most accurate means of diagnosing thyroid nodules, up to 40% of FNA samples are reported as “suspicious” or “indeterminate”, often resulting in more extensive surgery than would be necessary for benign or indolent tumors.

The present Example was designed to further characterize and improve a gene expression signature derived from a series of studies performed to discover genetic markers that differentiated thyroid cancer from benign thyroid tumors using genome-wide gene expression arrays. Starting with differentially expressed genes, 12 were characterized by immunohistochemical assays on tissue sections, a PCR-based assay of these genes was developed, and an initial expression signature of 3 genes was generated that achieved 84% specificity and 71% sensitivity in discriminating benign from malignant thyroid tumors.

In order to improve on these results, and taking advantage of the improving annotation of the human genome over the last decade, the present inventors first explored a broad range of splice variants of the original 12 gene set, with the goal of identifying variants that showed low or no expression in non-thyroid cells, such as white blood cells. This was critical since FNA samples typically are heavily contaminated with blood, and while this can be mitigated by antibody-coated magnetic bead purification steps, which was also explored in this project, each additional process leads to losses and decreases overall assay sensitivity.

The initial set of experiments was focused on testing a wide range on potentially diagnostic splice variants. This aspect was performed using samples from a frozen tissue bank, and allowed the identification of several promising gene transcript variants.

The next phase involved developing quantitative real-time PCR versions for these candidate transcripts and determining transcript abundance based on threshold PCR-cycle (Ct) rather than by semiquantitative gel-based assays. At the same time, several complementary approaches were developed to address the inherent issues associated with clinical FNA samples including dealing with the inevitable blood contamination, as well as variable and unpredictable numbers of thyroid-derived epithelial cells present in the samples. To address the former, both positive and negative selection strategies were used based on epithelial or WBC-specific antibody-coated magnetic beads. These were successful, but did negatively impact overall sensitivity, as expected for any purification method. Therefore, the present inventors also explored an alternative approach that avoided this step, but required two conditions to be successful: first, the selected gene expression markers had to remain at undetectable levels in contaminating WBCs; and second, a thyrocyte-specific, but universally expressed marker was needed to serve as thyrocyte load control. Several candidates were tested, and the TPO gene (thyroid peroxidase) was chosen for its consistent expression levels across a variety of histological thyroid tumors and benign conditions.

In the final phase of the present Example, qPCR of selected markers was performed on a cohort of samples from an archival FNA collection, as well as from freshly collected samples to test the approach in circumstances closely resembling the clinical setting.

TABLE 7

Gene transcripts and transcript variants studied.

| | | | |
|---|---|---|---|
| Symbol | Ref Gene | Gene Name | Transcript variants |
| c-KIT | NM_000222.2 | KIT proto-oncogene receptor tyrosine kinase | V1; total of V1 + V2 |
| CDH3 | NM_001793.5 | Cadherin 3, type 1, P-cadherin | V1+2+3+7; V1+2+3 |
| CEACAM6 | NM_002483.6 | CEA related cell adhesion molecule 6 | transcript variant 1 |
| DIRAS3 | NM_004675.3 | DIRAS family GTPase 3 | |
| DPP4 | NM_001935.3 | Dipeptidyl-peptidase 4 | V1 + V2 |
| FNDC4 | NM_022823.2 | Fibronectin type III domain containing 4 | |
| HMGA2 | NM_003483.4 | HMG AT-hook 2 | transcript variant 1 |
| KLK7 | NM_005046.3 | Kallikrein related peptidase 7 | transcript variant 1 |
| MRC2 | NM_006039.4 | Mannose receptor C type 2 | V1,3,4 |
| PLAG1 | NM_002655.2 | PLAG1 zinc finger | V1, and V 1 +2 |
| PRSS3 | NM_007343.3 | Serine protease 3 | V1+V3 |
| SFN | NM_006142.3 | Stratifin | |

FIGS. 7-8 show the data obtained testing various gene expression markers on thyroid tumor tissue samples of various subtypes. The tumor subtypes tested included: Adenomatoid Nodules (AN), Follicular Adenomas (FA), Encapsulated FVPTC (EFV), Follicular variant of PTC (FV), and Papillary thyroid cancer (PTC).

Of particular interest is the EFV subtype, also known as NIFTP, which is a new subcategory of tumors morphologically similar to FVPTC, but encapsulated and with no evidence of invasive behavior. Until the recent revision of the ATA treatment guidelines for thyroid neoplasia, this subtype was classified as malignant, often leading to total thyroidectomy. This has now been reclassified as benign lesion, with lesser surgical procedures and potentially simple observation as therapeutic options, which are currently under active investigation.

The present inventors favored a more extensive exploration of a wider set of candidate expression markers, including many potential splice variants, to optimize the ability to find signatures not only discriminating benign from malignant tumors, but specifically also of use in correctly classifying the new NIFTP subset of tumors, which all current commercially available molecular tests misclassify as malignant.

Therefore, the present inventors focused on signatures that ideally classified NIFTP cases as either intermediate or largely in the benign category, with the understanding that individual NIFTP could legitimately show results consistent with malignancy, as these may well be precursor lesions, for which a more aggressive therapeutic approach may be appropriate. As FIGS. 4-5 illustrate, several of the candidate markers and signatures score the NIPFP tumors in an intermediate range.

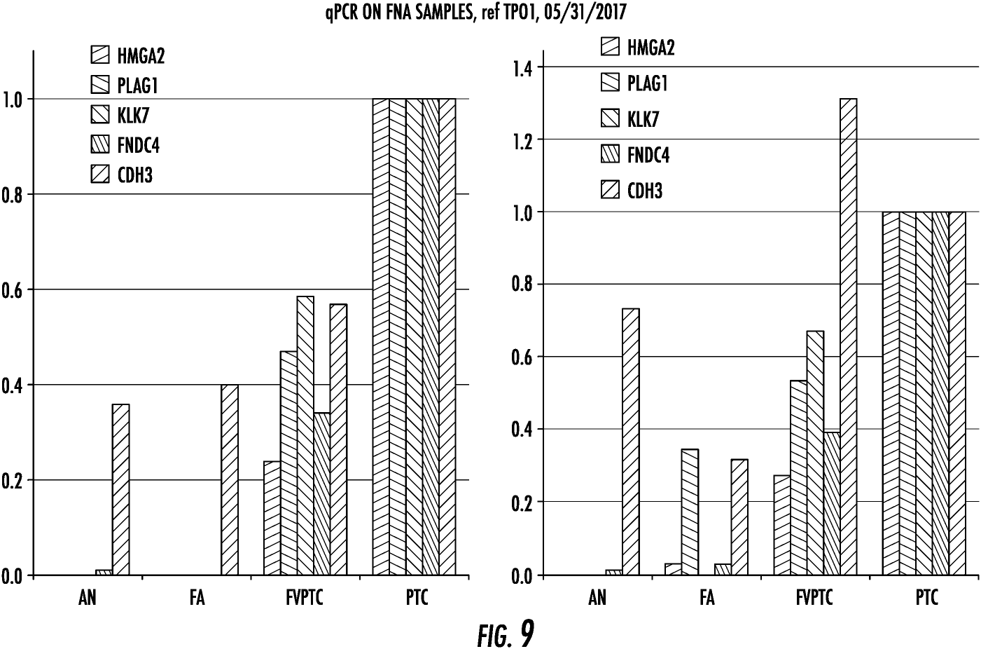

FIG. 9 illustrates the effect of shifting from the standard housekeeping gene GapDH to the thyrocyte-specific TPO gene as load control for the qPCR assays performed on FNA material. The most significant effect of this modification was to allow the removal of samples that would have been scored as false negative due to lack of signal, but were in fact devoid of thyroid epithelial cells.

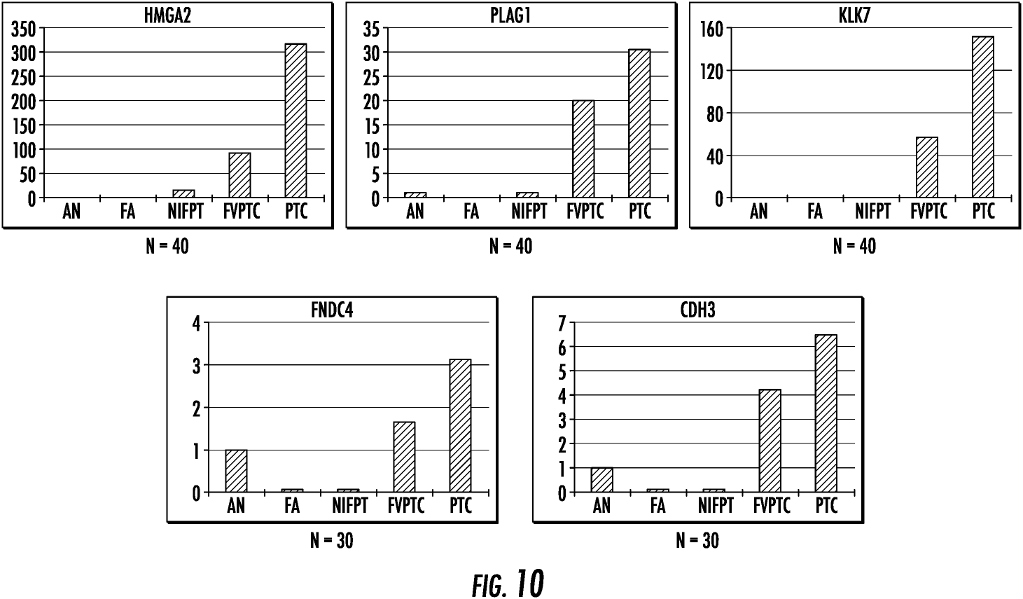

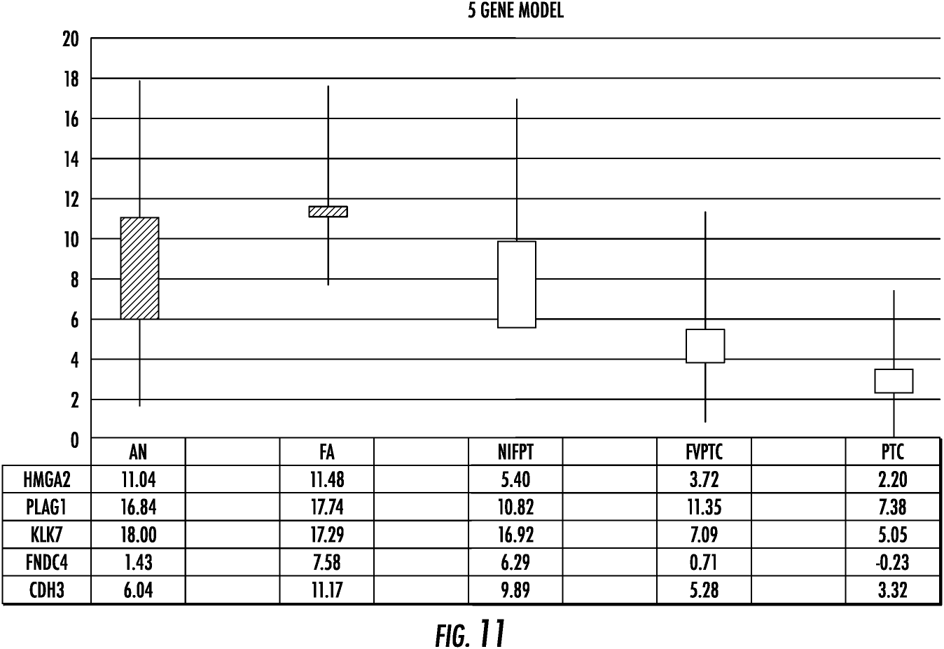

FIGS. 10-11 illustrate the results of the final set of diagnostic markers when tested on a validation cohort of archival FNA samples consisting of 22 benign and 18 malignant tumors, classifying the 6 NIFTP cases as benign. The problematic NIFTP class scores well within the benign tumor range, defined here simply as the average minus 1 standard deviation of AN & FA samples in this test cohort.

TABLE 8

| Diagnosis | True + | False + | True | False | |
|---|---|---|---|---|---|
| AN | | 2 | 7 | | |
| FA | | 0 | 7 | | Benign |
| NIFTP | | 1 | 5 | | 22 |
| FVPTC | 5 | | | 2 | Malignant |
| PTC | 10 | | | 1 | 18 |
| | 15 | 3 | 19 | 3 | 40 |

Using the approach indicated above, the present inventors achieved an overall assay sensitivity of 83% and a specificity of 86% in the 3-gene model, and almost identical results with a 5-gene model combining HMGA2, PLAG1, KLK7, FNDC4, and CDH3. This is remarkable because the experimental approach is directly applicable to the clinical setting, and includes a significant subset of typically misclassified NIFPT cases.

It is also worth noting that avoiding the need to enrich for thyroid cells in the archival FNA samples improved assay sensitivity from about 50% to 75%, based on a detectable TPO signal. This improved further to 90% on fresh FNA samples with magnetic bead enrichment, and the most recent experience on a limited number of fresh FNA samples avoiding bead enrichment resulted in no samples without detectable TPO signal so far.

That which is claimed:

1. A method for identifying a thyroid tumor from a patient as benign or malignant comprising the steps of:
(a) measuring expression, in a sample obtained from the patient, by real-time quantitative polymerase chain reaction (RT-qPCR) of at least three splice variant markers of a panel comprising high mobility group AT-hook 2 (HMGA2), transcript variant 1 (NCBI Reference Sequence, NM_003483.5); PLAG1 zine finger (PLAG1), transcript variant 1 (NCBI Reference Sequence, NM_002655.3); kallikrein related peptidase 7 (KLK7), transcript variant 1 (NCBI Reference Sequence, NM_005046.4); fibronectin type III domain containing 4 (FNDC4) (NCBI Reference Sequence, NM_022823.3); and cadherin 3 (CDH3), transcript variant 1 (NCBI Reference Sequence NM_001793.6); and
(b) identifying the tumor as benign or malignant based on the measured expression levels of the panel of splice variant markers as compared to a control.

2. The method of claim 1, wherein the sample is a fine needle aspiration (FNA) biopsy.

3. The method of claim 1, wherein the markers in the panel are not detectable in peripheral blood mononuclear cells.

4. The method of claim 1, further comprising detecting the presence of thyroid epithelial cells in the sample.

5. The method of claim 4, wherein the detecting step comprises measuring the expression of thyroid peroxidase isoform 1 (TPO1).

6. The method of claim 5, where the RT-qPCR is performed using primers that amplify all or a part of nucleotides 441-910 of SEQ ID NO:23 (TPO1).

7. The method of claim 6, wherein the primers comprise at least one of SEQ ID NOS:11-12.

8. The method of claim 1, wherein the RT-qPCR is performed using primers that amplify all or a part of the following regions of the markers: nucleotides 961-1320 of SEQ ID NO:13 (HMGA2); nucleotides 154-513 of SEQ ID NO:15 (PLAG1); nucleotides 204-563 of SEQ ID NO:17 (KLK7); nucleotides 481-800 of SEQ ID NO:19 (FNDC4); and nucleotides 767-1246 of SEQ ID NO:21 (CDH3).

9. The method of claim 8, wherein the HMGA2 primers comprise at least one of SEQ ID NOS:1-2.

10. The method of claim 8, wherein the PLAG1 primers comprise at least one of SEQ ID NOS:3-4.

11. The method of claim 8, wherein the KLK7 primers comprise at least one of SEQ ID NOS:5-6.

12. The method of claim 8, wherein the FNDC4 primers comprise at least one of SEQ ID NOS:7-8.

13. The method of claim 8, wherein the CDH3 primers comprise at least one of SEQ ID NOS:9-10.

14. The method of claim 1, wherein the sample is from a thyroid FNA previously determined to be indeterminate.

15. The method of claim 1, wherein the method distinguishes non-invasive follicular thyroid neoplasm with papillary-like nuclear features (NIFTP) from malignant follicular variant of papillary thyroid cancer (FVPTC).

16. The method of claim 1, wherein the identifying step comprises normalizing marker expression to TPO1; z-transforming to create a composite score; and performing a receiver operating characteristic (ROC) analysis.

17. The method of claim 1, further comprising treating a patient who is identified as having a malignant tumor with a thyroidectomy, hemithyroidectomy, radioactive iodine therapy, and combinations thereof.

18. The method of claim 17, wherein treatment further comprises one or more of a TERT inhibitor, a BRAF V600E inhibitor, a MEK inhibitor or combinations thereof,
the method of claim 1, wherein the treatment modality comprises administering to the subject both TERT inhibitor and BRAF V600E/MEK inhibitors.

19. A method for treating a patient having a malignant thyroid tumor or nodule comprising the step of performing one or more of a thyroidectomy, hemithyroidectomy, and radioactive iodine therapy and/or administering one or more of a TERT inhibitor, a BRAF V600E inhibitor, and a MEK inhibitor to a patient identified as having a malignant thyroid tumor based on expression of at least three of the following splice variant markers: HMGA2, transcript variant 1 (NCBI Reference Sequence, NM_003483.5); PLAG1, transcript variant 1 (NCBI Reference Sequence, NM_002655.3); KLK7, transcript variant 1 (NCBI Reference Sequence, NM_005046.4); FNDC4 (NCBI Reference Sequence, NM_022823.3); and CDH3, transcript variant 1 (NCBI Reference Sequence NM_001793.6).

20. A method for treating a patient having a malignant thyroid tumor or nodule comprising the steps of:
(a) measuring expression, in a sample obtained from the patient, by RT-qPCR of at least three splice variant markers of a panel comprising HMGA2, transcript variant 1 (NCBI Reference Sequence, NM_003483.5); PLAG1, transcript variant 1 (NCBI Reference Sequence, NM_002655.3); KLK7, transcript variant 1 (NCBI Reference Sequence, NM_005046.4); FNDC4 (NCBI Reference Sequence, NM_022823.3); and CDH3, transcript variant 1 (NCBI Reference Sequence NM_001793.6);
(b) identifying the tumor as malignant based on the measured expression levels of the panel of splice variant markers as compared to a control; and treating the patient with one or more of a thyroidectomy, hemithyroidectomy, radioactive iodine therapy, TERT inhibitor, BRAF V600E inhibitor, and MEK inhibitor.
